(19) United States
(12) Patent Application Publication
(10) Pub. No.: US 2024/0398567 A1
(43) Pub. Date: Dec. 5, 2024

(54) HEART VALVE REPAIR DEVICES AND DELIVERY DEVICES THEREFOR

(71) Applicant: EDWARDS LIFESCIENCES CORPORATION, Irvine, CA (US)

(72) Inventors: Grant Matthew Stearns, Costa Mesa, CA (US); Eric Michael Oberwise, Newport Beach, CA (US); Lauren R. Freschauf, Mission Viejo, CA (US)

(21) Appl. No.: 18/800,096

(22) Filed: Aug. 11, 2024

Related U.S. Application Data
(63) Continuation of application No. PCT/US2023/012802, filed on Feb. 10, 2023.
(60) Provisional application No. 63/310,557, filed on Feb. 15, 2022.

(51) Int. Cl. A61F 2/24

(52) U.S. Cl. CPC A61F 2/2463; A61F 2/246; A61F 2/2466; A61F 2220/0016; A61F 2220/0033; A61F 2250/0004

(57) ABSTRACT

A valve repair device for repairing a native valve of a patient. The valve repair device having one or more adjustable width paddle frame portions. The adjustable width paddle frame portions having an anterior portion extending from the medial side of the device to the lateral side of the device and a posterior portion extending from the medial side of the device and a lateral side of the device opposite the anterior portion. The anterior portion and the posterior portion are formed as a single frame member integrally joined at linking portions at the medial side of the device and a lateral side of the device. A connector includes attachment portions that connect to the linking portions to attach the adjustable width frame portion to the connector.

### RELATED APPLICATIONS

The present application is a continuation of Patent Cooperation Treaty application no. PCT/US2023/012802, filed on Feb. 10, 2023, which claims the benefit of U.S. Provisional Patent Application No. 63/310,557, filed on Feb. 15, 2022, which are both incorporated herein by reference in their entireties.

### BACKGROUND

The native heart valves (i.e., the aortic, pulmonary, tricuspid, and mitral valves) serve critical functions in assuring the forward flow of an adequate supply of blood through the cardiovascular system. These heart valves may be damaged, and thus rendered less effective, for example, by congenital malformations, inflammatory processes, infectious conditions, disease, etc. Such damage to the valves may result in serious cardiovascular compromise or death. Damaged valves may be surgically repaired or replaced during open heart surgery. However, open heart surgeries are highly invasive, and complications may occur. Transvascular techniques can be used to introduce and implant devices to treat a heart in a manner that is much less invasive than open heart surgery. As one example, a transvascular technique useable for accessing the native mitral and aortic valves is the trans-septal technique. The trans-septal technique comprises advancing a catheter into the right atrium (e.g., inserting a catheter into the right femoral vein, up the inferior vena cava and into the right atrium). The septum is then punctured, and the catheter passed into the left atrium. A similar transvascular technique can be used to implant a device within the tricuspid valve that begins similarly to the trans-septal technique but stops short of puncturing the septum and instead turns the delivery catheter toward the tricuspid valve in the right atrium.

A healthy heart has a generally conical shape that tapers to a lower apex. The heart is four-chambered and comprises the left atrium, right atrium, left ventricle, and right ventricle. The left and right sides of the heart are separated by a wall generally referred to as the septum. The native mitral valve of the human heart connects the left atrium to the left ventricle. The mitral valve has a very different anatomy than other native heart valves. The mitral valve includes an annulus portion, which is an annular portion of the native valve tissue surrounding the mitral valve orifice, and a pair of cusps, or leaflets, extending downward from the annulus into the left ventricle. The mitral valve annulus may form a “D”-shaped, oval, or otherwise out-of-round cross-sectional shape having major and minor axes. The anterior leaflet may be larger than the posterior leaflet, forming a generally “C”-shaped boundary between the abutting sides of the leaflets when they are closed together.

When operating properly, the anterior leaflet and the posterior leaflet function together as a one-way valve to allow blood to flow only from the left atrium to the left ventricle. The left atrium receives oxygenated blood from the pulmonary veins. When the muscles of the left atrium contract and the left ventricle dilates (also referred to as “ventricular diastole” or “diastole”), the oxygenated blood that is collected in the left atrium flows into the left ventricle. When the muscles of the left atrium relax and the muscles of the left ventricle contract (also referred to as “ventricular systole” or “systole”), the increased blood pressure in the left ventricle urges the sides of the two leaflets together, thereby closing the one-way mitral valve so that blood cannot flow back to the left atrium and is instead expelled out of the left ventricle through the aortic valve. To prevent the two leaflets from prolapsing under pressure and folding back through the mitral annulus toward the left atrium, a plurality of fibrous cords called chordae tendineae tether the leaflets to papillary muscles in the left ventricle.

Valvular regurgitation involves the valve improperly allowing some blood to flow in the wrong direction through the valve. For example, mitral regurgitation occurs when the native mitral valve fails to close properly and blood flows into the left atrium from the left ventricle during the systolic phase of heart contraction. Mitral regurgitation is one of the most common forms of valvular heart disease. Mitral regurgitation may have many different causes, such as leaflet prolapse, dysfunctional papillary muscles, stretching of the mitral valve annulus resulting from dilation of the left ventricle, more than one of these, etc. Mitral regurgitation at a central portion of the leaflets can be referred to as central jet mitral regurgitation and mitral regurgitation nearer to one commissure (i.e., location where the leaflets meet) of the leaflets can be referred to as eccentric jet mitral regurgitation. Central jet regurgitation occurs when the edges of the leaflets do not meet in the middle and thus the valve does not close, and regurgitation is present. Tricuspid regurgitation may be similar, but on the right side of the heart.

### SUMMARY

This summary is meant to provide some examples and is not intended to be limiting of the scope of the invention in any way. For example, any feature included in an example of this summary is not required by the claims, unless the claims explicitly recite the feature. Also, the features, components, steps, concepts, etc. described in examples in this summary and elsewhere in this disclosure can be combined in a variety of ways. Various features and steps as described elsewhere in this disclosure can be included in the examples summarized here.

In some implementations, there is provided an implantable device or implant (e.g., implantable device, etc.) that is configured to be positioned within a native heart valve to allow the native heart valve to form a more effective seal.

In some implementations, an implantable device or implant includes an anchor portion. Each anchor includes a plurality of paddles that are each moveable between an open position and a closed position.

In some implementations, an example valve repair device includes a pair of paddles movable between an open position and a closed position to attach the valve repair device to the native valve of the patient and one or more paddle frames configured to open and close the pair of paddles. The paddle frames can have an optional inner frame portion extending from a proximal portion of the device to a distal portion of the device, and an adjustable width frame portion having an anterior portion extending from the medial side of the device to the lateral side of the device and a posterior portion extending from the medial side of the device and a lateral side of the device opposite the anterior portion.

In some implementations, the anterior portion and the posterior portion are formed as a single frame member integrally joined at linking portions at the medial side of the device and a lateral side of the device.

In some implementations, a connector extends from the distal portion toward the proximal portion along both a medial side of the device and a lateral side of the device. The connector includes attachment portions that connect to the linking portions to attach the adjustable width frame portion to the connector.

In some implementations, the linking portions are configured as closed-ended projections.

In some implementations, each of the attachment portions of the connector include a hole through which a respective one of the closed-ended projections is received.

In some implementations, the anterior portion and the posterior portion of the adjustable width frame portion attach to one, or both, of the inner frame portion and the pair of paddles at the proximal portion of the device.

In some implementations, the adjustable width frame portion includes a shape-memory alloy.

In some implementations, the adjustable width frame portion is symmetric.

In some implementations, an example valve repair device includes a pair of paddles movable between an open position and a closed position. The pair of paddles are configured to attach the valve repair device to the native valve of the patient. One or more paddle frames can include an optional inner frame portion extending from a proximal portion of the device to a distal portion of the device. The inner frame portion is connected to the pair of paddles at the proximal portion.

In some implementations, a first adjustable width frame portion of a valve repair device has a first anterior portion extending from the medial side of the device to adjacent of the device and a first posterior portion extending from the medial side of the device to adjacent the midline of the device opposite the first anterior portion. In some implementations, second adjustable width frame portion has a second anterior portion extending from the lateral side of the device to adjacent the midline of the device and a second posterior portion extending from the lateral side of the device to adjacent the midline of the device opposite the second anterior portion.

In some implementations, the first anterior portion and the first posterior portion are integrally joined at a first linking portion at the medial side of the device and the second anterior portion and the second posterior portion are integrally joined at a second linking portion at the lateral side of the device.

In some implementations, a connector extends from the distal portion toward the proximal portion along both a medial side of the device and a lateral side of the device. In some implementations, the connector includes a first attachment portion that connects to the first linking portion and a second attachment portion that connects to the second linking portion to attach the adjustable width frame portion to the connector.

In some implementations, the first and second linking portions are configured as closed-ended projections.

In some implementations, the first and second attachment portions of the connector include a hole through which a respective one of the closed-ended projections is received.

In some implementations, the first anterior portion and the first posterior portion attach to one, or both, of the inner frame portion and the pair of paddles at the proximal end of the device.

In some implementations, each of the first adjustable width frame portion and the second adjustable width frame portion includes a shape-memory alloy.

In some implementations, the first adjustable width frame portion is symmetric.

In some implementations, an example valve repair device includes a pair of paddles movable between an open position and a closed position. The pair of paddles are configured to attach the valve repair device to the native valve of the patient. One or more paddle frames can include an optional inner frame portion extending from a proximal portion of the device to a distal portion of the device. The optional inner frame portion can be connected to the pair of paddles at the proximal portion.

In some implementations, a medial adjustable width frame portion is formed as a closed-loop extending from the medial side of the device to the lateral side of the device, and a posterior, adjustable width frame portion is formed as a closed-loop extending from the medial side of the device to the lateral side of the device and opposite the anterior, adjustable width frame portion.

In some implementations, a connector extends from the distal portion toward the proximal portion along both an anterior side of the device and a posterior side of the device. The connector includes a first attachment portion that connects to an anterior distal portion of the anterior, adjustable width frame portion. The connector includes a second attachment portion that connects to a posterior distal portion of the posterior, adjustable width frame portion.

In some implementations, the anterior distal portion of the anterior, adjustable width frame portion and the posterior distal portion of the posterior, adjustable width frame portion are laterally-extending, linear sections.

In some implementations, the first attachment portion of the connector includes a hole through which the posterior distal portion is received.

In some implementations, the anterior, adjustable width frame portion and the posterior, adjustable width frame portion attach to one, or both, of the inner frame portion and the pair of paddles at the proximal portion of the device.

In some implementations, each of the anterior, adjustable width frame portion and the posterior, adjustable width frame portion includes a shape-memory alloy.

In some implementations, the anterior, adjustable width frame portion and the posterior, adjustable width frame portion are symmetric.

In some implementations, the inner frame portion includes an anterior inner frame portion and a posterior inner frame portion separate from the anterior inner frame portion.

In some implementations, the anterior inner frame portion is integrally formed with the anterior, adjustable width frame portion.

In some implementations, the anterior inner frame portion is integrally connected with the anterior, adjustable width frame portion adjacent the proximal portion of the device.

In some implementations, the valve repair device can include an inner paddle and an outer paddle. The first attachment portion can connect to the anterior distal portion of the anterior, adjustable width frame portion at a position anteriorly outward of the outer paddle.

In some implementations, the device can comprise an inner paddle and an outer paddle. The first attachment portion can connect to the anterior distal portion of the anterior, adjustable width frame portion at a position anteriorly outward of the inner paddle and anteriorly inward of the outer paddle.

Any of the above systems, assemblies, devices, apparatuses, components, etc. can be sterilized (e.g., with heat, radiation, ethylene oxide, hydrogen peroxide, etc.) to ensure they are safe for use with patients, and any methods herein can comprise (or additional methods comprise or consist of) sterilization of one or more systems, devices, apparatuses, components, etc. herein (e.g., with heat, radiation, ethylene oxide, hydrogen peroxide, etc.).

A further understanding of the nature and advantages of the present invention are set forth in the following description and claims, particularly when considered in conjunction with the accompanying drawings in which like parts bear like reference numerals.

### BRIEF DESCRIPTION OF THE DRAWINGS

To further clarify various aspects of implementations of the present disclosure, a more particular description of the certain examples and implementations will be made by reference to various aspects of the appended drawings. These drawings depict only example implementations of the present disclosure and are therefore not to be considered limiting of the scope of the disclosure. Moreover, while the FIGS. can be drawn to scale for some examples, the FIGS. are not necessarily drawn to scale for all examples. Examples and other features and advantages of the present disclosure will be described and explained with additional specificity and detail through the use of the accompanying drawings in which:

FIG. 1 illustrates a cutaway view of the human heart in a diastolic phase;

FIG. 2 illustrates a cutaway view of the human heart in a systolic phase;

FIG. 3 illustrates a cutaway view of the human heart in a systolic phase showing mitral regurgitation;

FIG. 4 is the cutaway view of FIG. 3 annotated to illustrate a natural shape of mitral valve leaflets in the systolic phase;

FIG. 5 illustrates a healthy mitral valve with the leaflets closed as viewed from an atrial side of the mitral valve;

FIG. 6 illustrates a dysfunctional mitral valve with a visible gap between the leaflets as viewed from an atrial side of the mitral valve;

FIG. 7 illustrates a tricuspid valve viewed from an atrial side of the tricuspid valve;

FIGS. 8-14 show an example of an implantable device or implant, in various stages of deployment;

FIG. 15 shows an example of an implantable device or implant that is similar to the device illustrated by FIGS. 8-14, but where the paddles are independently controllable;

FIGS. 16-21 show the example implantable device or implant of FIGS. 8-14 being delivered and implanted within a native valve;

FIG. 22 shows a perspective view of an example implantable device or implant in a closed position;

FIG. 23 shows a perspective view of an example implantable device or implant in a closed position;

FIG. 24 illustrates an example valve repair device with paddles in an open position;

FIG. 25A illustrates another example valve repair device with paddles in a closed position;

FIG. 25B illustrates a top view of an example valve repair device;

FIG. 26 illustrates a perspective view of an example implantable device having paddles of adjustable widths;

FIG. 27 is a cross-section of the implantable device of FIG. 26 in which the implantable device is bisected;

FIG. 28 is another cross-section of the implantable device of FIG. 26 in which the implantable device is bisected along a plane perpendicular to the plane illustrated in FIG. 28;

FIG. 29 is a schematic illustration of an example implant catheter assembly coupled to an implantable device in which an actuation element is coupled to a paddle actuation control and to a driver head of the implantable device;

FIG. 30 is an illustration of the assembly of FIG. 29 with the implantable device rotated 90 degrees to show the paddle width adjustment element coupled to an inner end of a connector of the implantable device and coupled to a paddle width control;

FIG. 31 illustrates a perspective view of an example implantable device having paddles of adjustable widths;

FIG. 32A illustrates a perspective view of an example adjustable width frame member useable as part of the implantable device of FIG. 31;

FIG. 32B illustrates a top view of the adjustable width frame member of FIG. 32A in a flat, planar configuration;

FIG. 33 illustrates a top view of the adjustable width frame member of FIG. 32A;

FIG. 34 illustrates a front view of the adjustable width frame member of FIG. 32A;

FIG. 35 illustrates a top view of the adjustable width frame member of FIG. 32A connected to an example connector of the implantable device of FIG. 31;

FIG. 36 illustrates a front view of the adjustable width frame member and connector of FIG. 35;

FIG. 37 illustrates a perspective view of an example adjustable width frame member of usable as part of the implantable device of FIG. 31;

FIG. 38 illustrates a perspective view of an example implantable device having paddles of adjustable widths;

FIG. 39 illustrates a perspective view of an example adjustable width frame member useable as part of the implantable device of FIG. 38;

FIG. 40 illustrates a top view of the adjustable width frame member of FIG. 38;

FIG. 41 illustrates a front view of the adjustable width frame member of FIG. 38;

FIG. 42 illustrates a perspective view of the adjustable width frame member and a connector that can be the same as or similar to the connector of FIG. 35;

FIG. 43 illustrates a perspective view of an example implantable device having paddles of adjustable widths;

FIG. 44 illustrates a front view of the implantable device of FIG. 43;

FIG. 45 illustrates a side view of the implantable device of FIG. 43;

FIG. 46 illustrates a front view of an example implantable device having paddles of adjustable widths;

FIG. 47 illustrates a perspective view of an example paddle frame useable as part of an implantable device;

FIG. 48 illustrates a side view of the paddle frame of FIG. 46; and

FIG. 49 illustrates a front view of the paddle frame of FIG. 46.

### DETAILED DESCRIPTION

The following description refers to the accompanying drawings, which illustrate example implementations of the present disclosure. Other implementations having different structures and operation do not depart from the scope of the present disclosure.

Some implementations of the present disclosure are directed to systems, devices, methods, etc. for repairing a defective heart valve. For example, some implementations of valve repair devices, implantable devices, implants, and systems (including systems for delivery thereof) are disclosed herein, and any combination of these options can be made unless specifically excluded. In other words, individual components of the disclosed devices and systems can be combined unless mutually exclusive or otherwise physically impossible. Further, the techniques and methods herein can be performed on a living animal or on a simulation, such as on a cadaver, cadaver heart, simulator (e.g., with the body parts, heart, tissue, etc. being simulated), imaginary person (e.g., demonstrating in the air on an imaginary heart), etc.

As described herein, when one or more components are described as being connected, joined, affixed, coupled, attached, or otherwise interconnected, such interconnection can be direct as between the components or can be indirect such as through the use of one or more intermediary components. Also as described herein, reference to a “member,” “component,” or “portion” shall not be limited to a single structural member, component, or element but can include an assembly of components, members, or elements. Also as described herein, the terms “substantially” and “about” are defined as at least close to (and includes) a given value or state (preferably within 10% of, more preferably within 1% of, and most preferably within 0.1% of).

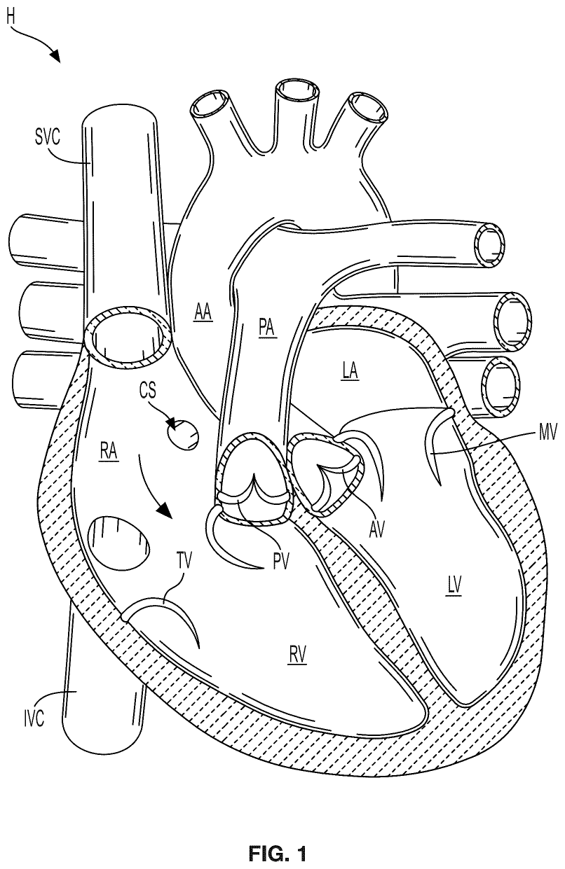

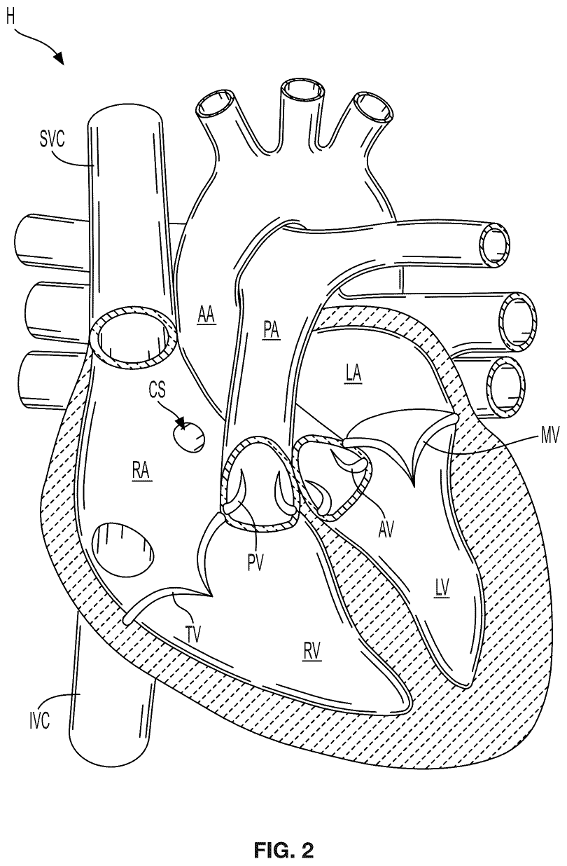

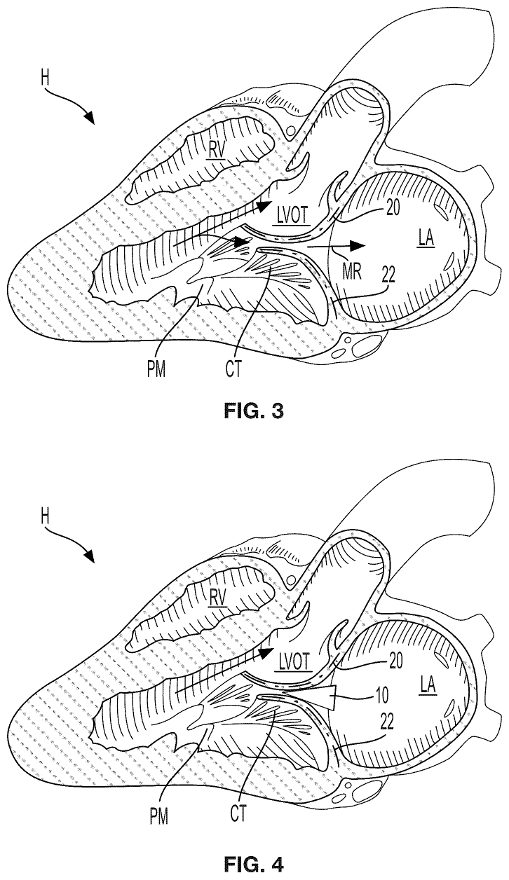

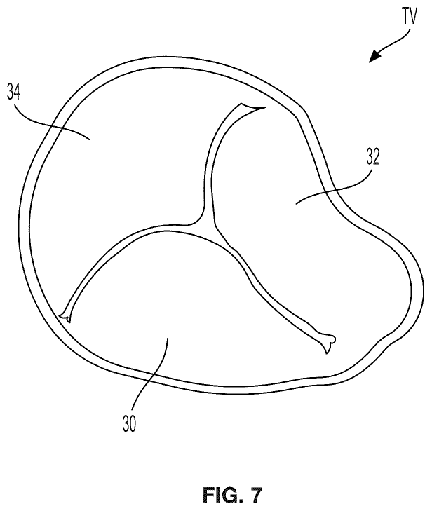

FIGS. 1 and 2 are cutaway views of the human heart H in diastolic and systolic phases, respectively. The right ventricle RV and left ventricle LV are separated from the right atrium RA and left atrium LA, respectively, by the tricuspid valve TV and mitral valve MV; i.e., the atrioventricular valves. Additionally, the aortic valve AV separates the left ventricle LV from the ascending aorta AA, and the pulmonary valve PV separates the right ventricle from the pulmonary artery PA. Each of these valves has flexible leaflets (e.g., leaflets 20, 22 shown in FIGS. 3-6 and leaflets 30, 32, 34 shown in FIG. 7) extending inward across the respective orifices that come together or “coapt” in the flow stream to form the one-way, fluid-occluding surfaces. The native valve repair systems of the present application are frequently described and/or illustrated with respect to the mitral valve MV. Therefore, anatomical structures of the left atrium LA and left ventricle LV will be explained in greater detail. However, the devices described herein can also be used in repairing other native valves, e.g., the devices can be used in repairing the tricuspid valve TV, the aortic valve AV, and the pulmonary valve PV.

The left atrium LA receives oxygenated blood from the lungs. During the diastolic phase, or diastole, seen in FIG. 1, the blood that was previously collected in the left atrium LA (during the systolic phase) moves through the mitral valve MV and into the left ventricle LV by expansion of the left ventricle LV. In the systolic phase, or systole, seen in FIG. 2, the left ventricle LV contracts to force the blood through the aortic valve AV and ascending aorta AA into the body. During systole, the leaflets of the mitral valve MV close to prevent the blood from regurgitating from the left ventricle LV and back into the left atrium LA and blood is collected in the left atrium from the pulmonary vein. In some implementations, the devices described by the present application are used to repair the function of a defective mitral valve MV. That is, the devices are configured to help close the leaflets of the mitral valve to prevent, inhibit or reduce blood from regurgitating from the left ventricle LV and back into the left atrium LA. Many of the devices described in the present application are designed to easily grasp and secure the native leaflets around a coaptation element or spacer that beneficially acts as a filler in the regurgitant orifice to prevent or inhibit back flow or regurgitation during systole, though this is not necessary.

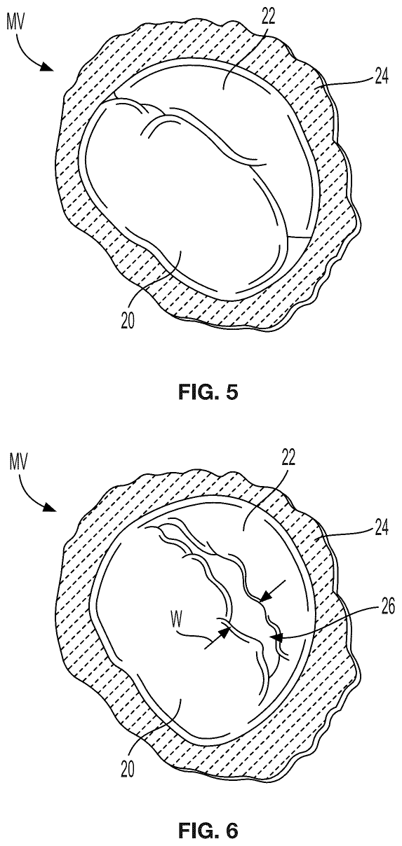

Referring now to FIGS. 1-7, the mitral valve MV includes two leaflets, the anterior leaflet 20 and the posterior leaflet 22. The mitral valve MV also includes an annulus 24 (see FIG. 5), which is a variably dense fibrous ring of tissues that encircles the leaflets 20, 22. Referring to FIGS. 3 and 4, the mitral valve MV is anchored to the wall of the left ventricle LV by chordae tendineae CT. The chordae tendineae CT are cord-like tendons that connect the papillary muscles PM (i.e., the muscles located at the base of the chordae tendineae CT and within the walls of the left ventricle LV) to the leaflets 20, 22 of the mitral valve MV. The papillary muscles PM serve to limit the movements of leaflets 20, 22 of the mitral valve MV and prevent the mitral valve MV from being reverted. The mitral valve MV opens and closes in response to pressure changes in the left atrium LA and the left ventricle LV. The papillary muscles PM do not open or close the mitral valve MV. Rather, the papillary muscles PM support or brace the leaflets 20, 22 against the high pressure needed to circulate blood throughout the body. Together the papillary muscles PM and the chordae tendineae CT are known as the subvalvular apparatus, which functions to keep the mitral valve MV from prolapsing into the left atrium LA when the mitral valve closes. As seen from a Left Ventricular Outflow Tract (LVOT) view shown in FIG. 3, the anatomy of the leaflets 20, 22 is such that the inner sides of the leaflets coapt at the free end portions and the leaflets 20, 22 start receding or spreading apart from each other. The leaflets 20, 22 spread apart in the atrial direction, until each leaflet meets with the mitral annulus.

Various disease processes can impair proper function of one or more of the native valves of the heart H. These disease processes include degenerative processes (e.g., Barlow's Disease, fibroelastic deficiency, etc.), inflammatory processes (e.g., Rheumatic Heart Disease), and infectious processes (e.g., endocarditis, etc.). In addition, damage to the left ventricle LV or the right ventricle RV from prior heart attacks (i.e., myocardial infarction secondary to coronary artery disease) or other heart diseases (e.g., cardiomyopathy, etc.) may distort a native valve's geometry, which may cause the native valve to dysfunction. However, the majority of patients undergoing valve surgery, such as surgery to the mitral valve MV, suffer from a degenerative disease that causes a malfunction in a leaflet (e.g., leaflets 20, 22) of a native valve (e.g., the mitral valve MV), which results in prolapse and regurgitation.

Generally, a native valve may malfunction in different ways: including (1) valve stenosis; and (2) valve regurgitation. Valve stenosis occurs when a native valve does not open completely and thereby causes an obstruction of blood flow. Typically, valve stenosis results from buildup of calcified material on the leaflets of a valve, which causes the leaflets to thicken and impairs the ability of the valve to fully open to permit forward blood flow. Valve regurgitation occurs when the leaflets of the valve do not close completely thereby causing blood to leak back into the prior chamber (e.g., causing blood to leak from the left ventricle to the left atrium).

There are three main mechanisms by which a native valve becomes regurgitant—or incompetent—which include Carpentier's type I, type II, and type III malfunctions. A Carpentier type I malfunction involves the dilation of the annulus such that normally functioning leaflets are distracted from each other and fail to form a tight seal (i.e., the leaflets do not coapt properly). Included in a type I mechanism malfunction are perforations of the leaflets, as are present in endocarditis. A Carpentier's type II malfunction involves prolapse of one or more leaflets of a native valve above a plane of coaptation. A Carpentier's type III malfunction involves restriction of the motion of one or more leaflets of a native valve such that the leaflets are abnormally constrained below the plane of the annulus. Leaflet restriction may be caused by rheumatic disease or dilation of a ventricle.

Referring to FIG. 5, when a healthy mitral valve MV is in a closed position, the anterior leaflet 20 and the posterior leaflet 22 coapt, which prevents blood from leaking from the left ventricle LV to the left atrium LA. Referring to FIGS. 3 and 6, mitral regurgitation MR occurs when the anterior leaflet 20 and/or the posterior leaflet 22 of the mitral valve MV is displaced into the left atrium LA during systole so that the edges of the leaflets 20, 22 are not in contact with each other. This failure to coapt causes a gap 26 between the anterior leaflet 20 and the posterior leaflet 22, which allows blood to flow back into the left atrium LA from the left ventricle LV during systole, as illustrated by the mitral regurgitation MR flow path shown in FIG. 3. Referring to FIG. 6, the gap 26 can have a width W between about 2.5 mm and about 17.5 mm, between about 5 mm and about 15 mm, between about 7.5 mm and about 12.5 mm, or about 10 mm. In some situations, the gap 26 can have a width W greater than 15 mm or even 17.5 mm. As set forth above, there are several different ways that a leaflet (e.g., leaflets 20, 22 of mitral valve MV) may malfunction which can thereby lead to valvular regurgitation.

In any of the above-mentioned situations, a valve repair device or implant is desired that is capable of engaging the anterior leaflet 20 and the posterior leaflet 22 to close the gap 26 and prevent or inhibit regurgitation of blood through the mitral valve MV. As can be seen in FIG. 4, an abstract representation of an implantable device, valve repair device, or implant 10 is shown implanted between the leaflets 20, 22 such that regurgitation does not occur during systole (compare FIG. 3 with FIG. 4). In some implementations, the coaptation element (e.g., spacer, coaption element, gap filler, etc.) of the device 10 has a generally tapered or triangular shape that naturally adapts to the native valve geometry and to its expanding leaflet nature (toward the annulus). In this application, the terms spacer, coaption element, coaptation element, and gap filler are used interchangeably and refer to an element that fills a portion of the space between native valve leaflets and/or that is configured such that the native valve leaflets engage or “coapt” against (e.g., such that the native leaflets coapt against the coaption element, coaptation element, spacer, etc. instead of only against one another). The coaptation element or spacer is optional in any of the valve repair devices disclosed herein.

Although stenosis or regurgitation may affect any valve, stenosis is predominantly found to affect either the aortic valve AV or the pulmonary valve PV, and regurgitation is predominantly found to affect either the mitral valve MV or the tricuspid valve TV. Both valve stenosis and valve regurgitation increase the workload of the heart H and may lead to very serious conditions if left un-treated; such as endocarditis, congestive heart failure, permanent heart damage, cardiac arrest, and ultimately death. Because the left side of the heart (i.e., the left atrium LA, the left ventricle LV, the mitral valve MV, and the aortic valve AV) are primarily responsible for circulating the flow of blood throughout the body. Accordingly, because of the substantially higher pressures on the left side heart dysfunction of the mitral valve MV or the aortic valve AV is particularly problematic and often life threatening.

Malfunctioning native heart valves can either be repaired or replaced. Repair typically involves the preservation and correction of the patient's native valve. Replacement typically involves replacing the patient's native valve with a biological or mechanical substitute. Typically, the aortic valve AV and pulmonary valve PV are more prone to stenosis. Because stenotic damage sustained by the leaflets is irreversible, treatments for a stenotic aortic valve or stenotic pulmonary valve can be removal and replacement of the valve with a surgically implanted heart valve, or displacement of the valve with a transcatheter heart valve. The mitral valve MV and the tricuspid valve TV are more prone to deformation of leaflets and/or surrounding tissue, which, as described above, may prevent the mitral valve MV or tricuspid valve TV from closing properly and allows for regurgitation or back flow of blood from the ventricle into the atrium (e.g., a deformed mitral valve MV may allow for regurgitation or back flow from the left ventricle LV to the left atrium LA as shown in FIG. 3). The regurgitation or back flow of blood from the ventricle to the atrium results in valvular insufficiency. Deformations in the structure or shape of the mitral valve MV or the tricuspid valve TV are often repairable. In addition, regurgitation may occur due to the chordae tendineae CT becoming dysfunctional (e.g., the chordae tendineae CT may stretch or rupture), which allows the anterior leaflet 20 and the posterior leaflet 22 to be reverted such that blood is regurgitated into the left atrium LA. The problems occurring due to dysfunctional chordae tendineae CT can be repaired by repairing the chordae tendineae CT or the structure of the mitral valve MV (e.g., by securing the leaflets 20, 22 at the affected portion of the mitral valve).

The devices and procedures disclosed herein often make reference to repairing the structure of a mitral valve. However, it should be understood that the devices and concepts provided herein can be used to repair any native valve, as well as any component of a native valve. Such devices can be used between the leaflets 20, 22 of the mitral valve MV to prevent or inhibit regurgitation of blood from the left ventricle into the left atrium. With respect to the tricuspid valve TV (FIG. 7), any of the devices and concepts herein can be used between any two of the anterior leaflet 30, septal leaflet 32, and posterior leaflet 34 to prevent or inhibit regurgitation of blood from the right ventricle into the right atrium. In addition, any of the devices and concepts provided herein can be used on all three of the leaflets 30, 32, 34 together to prevent or inhibit regurgitation of blood from the right ventricle to the right atrium. That is, the valve repair devices or implants provided herein can be centrally located between the three leaflets 30, 32, 34.

An example implantable device or implant can optionally have a coaptation element (e.g., spacer, coaption element, gap filler, etc.) and at least one anchor (e.g., one, two, three, or more). In some implementations, an implantable device or implant can have any combination or sub-combination of the features disclosed herein without a coaptation element. When included, the coaptation element (e.g., spacer, coaption element, gap filler, etc.) is configured to be positioned within the native heart valve orifice to help fill the space between the leaflets and form a more effective seal, thereby reducing or preventing or inhibiting regurgitation described above. The coaptation element can have a structure that is impervious to blood (or that resists blood flow therethrough) and that allows the native leaflets to close around the coaptation element during ventricular systole to block blood from flowing from the left or right ventricle back into the left or right atrium, respectively. The device or implant can be configured to seal against two or three native valve leaflets; that is, the device can be used in the native mitral (bicuspid) and tricuspid valves. The coaptation element is sometimes referred to herein as a spacer because the coaptation element can fill a space between improperly functioning native leaflets (e.g., mitral leaflets 20, 22 or tricuspid leaflets 30, 32, 34) that do not close completely.

The optional coaptation element (e.g., spacer, coaptation element, gap filler, etc.) can have various shapes. In some implementations, the coaptation element can have an elongated cylindrical shape having a round cross-sectional shape. In some implementations, the coaptation element can have an oval cross-sectional shape, an ovoid cross-sectional shape, a crescent cross-sectional shape, a rectangular cross-sectional shape, or various other non-cylindrical shapes. In some implementations, the coaptation element can have an atrial portion positioned in or adjacent to the atrium, a ventricular or lower portion positioned in or adjacent to the ventricle, and a side surface that extends between the native leaflets. In some implementations configured for use in the tricuspid valve, the atrial or upper portion is positioned in or adjacent to the right atrium, and the ventricular or lower portion is positioned in or adjacent to the right ventricle, and the side surfaces extend between the native tricuspid leaflets.

In some implementations, the anchor can be configured to secure the device to one or both of the native leaflets such that the coaptation element is positioned between the two native leaflets. In some implementations configured for use in the tricuspid valve, the anchor is configured to secure the device to one, two, or three of the tricuspid leaflets such that the coaptation element is positioned between the three native leaflets. In some implementations, the anchor can attach to the coaptation element at a location adjacent the ventricular portion of the coaptation element. In some implementations, the anchor can attach to an actuation element (e.g., an actuation shaft, actuation tube, actuation wire, etc.) to which the coaptation element is also attached. In some implementations, the anchor and the coaptation element can be positioned independently with respect to each other by separately moving each of the anchor and the coaptation element along the longitudinal axis of the actuation element (e.g., actuation shaft, actuation rod, actuation tube, actuation wire, etc.). In some implementations, the anchor and the coaptation element can be positioned simultaneously by moving the anchor and the coaptation element together along the longitudinal axis of the actuation element (e.g., shaft, actuation wire, etc.). The anchor can be configured to be positioned behind a native leaflet when implanted such that the leaflet is grasped by the anchor.

The device or implant can be configured to be implanted via a delivery system or other means for delivery. The delivery system can comprise one or more of a guide/delivery sheath, a delivery catheter, a steerable catheter, an implant catheter, tube, combinations of these, etc. The coaptation element and the anchor can be compressible to a radially compressed state and can be self-expandable to a radially expanded state when compressive pressure is released. The device can be configured for the anchor to be expanded radially away from the still-compressed coaptation element initially in order to create a gap between the coaptation element and the anchor. A native leaflet can then be positioned in the gap. The coaptation element can be expanded radially, closing the gap between the coaptation element and the anchor and capturing the leaflet between the coaptation element and the anchor. In some implementations, the anchor and coaptation element are optionally configured to self-expand. The implantation methods for some implementations can be different and are more fully discussed below with respect to each implementation. Additional information regarding these and other delivery methods can be found in U.S. Pat. No. 8,449,599 and U.S. Patent Application Publication Nos. 2014/0222136, 2014/0067052, 2016/0331523, and PCT patent application publication Nos. WO2020/076898, each of which is incorporated herein by reference in its entirety for all purposes. These method(s) can be performed on a living animal or on a simulation, such as on a cadaver, cadaver heart, simulator (e.g., with the body parts, heart, tissue, etc. being simulated), etc. mutatis mutandis.

The disclosed devices or implants can be configured such that the anchor is connected to a leaflet, taking advantage of the tension from native chordae tendineae to resist high systolic pressure urging the device toward the left atrium. During diastole, the devices can rely on the compressive and retention forces exerted on the leaflet that is grasped by the anchor.

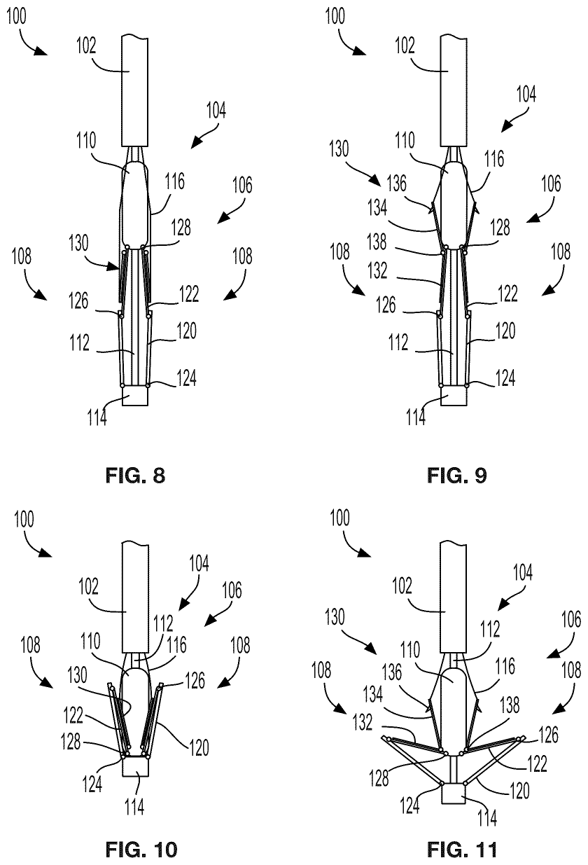

Referring now to FIGS. 8-15, a schematically illustrated implantable device or implant 100 (e.g., an implantable prosthetic device, a prosthetic spacer device, a valve repair device, etc.) is shown in various stages of deployment. The device or implant 100 and other similar devices/implants are described in more detail in PCT patent application publication Nos. WO2018/195215, WO2020/076898, and WO 2019/139904, which are incorporated herein by reference in their entirety. The device 100 can include any other features for an implantable device or implant discussed in the present application or the applications cited above, and the device 100 can be positioned to engage valve tissue (e.g., leaflets 20, 22, 30, 32, 34) as part of any suitable valve repair system (e.g., any valve repair systems/devices disclosed in the present application or the applications cited above).

The device or implant 100 is deployed from a delivery system 102. The delivery system 102 can comprise one or more of a catheter, a sheath, a guide catheter/sheath, a delivery catheter/sheath, a steerable catheter, an implant catheter, a tube, a channel, a pathway, combinations of these, etc. The device or implant 100 includes a coaptation portion 104 and an anchor portion 106.

In some implementations, the coaptation portion 104 of the device or implant 100 includes a coaptation element 110 that is adapted to be implanted between leaflets of a native valve (e.g., a native mitral valve, native tricuspid valve, etc.) and is slidably attached to an actuation element 112 (e.g., actuation wire, actuation shaft, actuation tube, etc.). The anchor portion 106 includes one or more anchors 108 that are actuatable between open and closed conditions and can take a wide variety of forms, such as, for example, paddles, gripping elements, or the like. Actuation of the actuation element 112 opens and closes the anchor portion 106 of the device 100 to grasp the native valve leaflets during implantation. The actuation element 112 (as well as other actuation elements disclosed herein) can take a wide variety of different forms (e.g., as a wire, rod, shaft, tube, screw, suture, line, strip, combination of these, etc.), be made of a variety of different materials, and have a variety of configurations. As one example, the actuation element can be threaded such that rotation of the actuation element moves the anchor portion 106 relative to the coaptation portion 104. Or, the actuation element can be unthreaded, such that pushing or pulling the actuation element 112 moves the anchor portion 106 relative to the coaptation portion 104.

The anchor portion 106 and/or anchors of the device 100 include outer paddles 120 and inner paddles 122 that are, in some implementations, connected between a cap 114 and a coaptation element 110 by portions 124, 126, 128. The portions 124, 126, 128 can be jointed and/or flexible to move between all of the positions described below. The interconnection of the outer paddles 120, the inner paddles 122, the coaptation element 110, and the cap 114 by the portions 124, 126, and 128 can constrain the device to the positions and movements illustrated herein.

In some implementations, the delivery system 102 includes a steerable catheter, implant catheter, and the actuation element 112 (e.g., actuation wire, actuation shaft, etc.). These can be configured to extend through a guide catheter/sheath (e.g., a transseptal sheath, etc.). In some implementations, the actuation element 112 extends through a delivery catheter and the coaptation element 110 to the distal end (e.g., a cap 114 or other attachment portion at the distal connection of the anchor portion 106). Extending and retracting the actuation element 112 increases and decreases the spacing between the coaptation element 110 and the distal end of the device (e.g., the cap 114 or other attachment portion), respectively. In some implementations, a collar or other attachment element (e.g., coupler, clamp, removable fastener, clip, etc.) removably attaches the coaptation element 110 to the delivery system 102, either directly or indirectly, so that the actuation element 112 slides through the collar or other attachment element and, in some implementations, through a coaptation element 110 during actuation to open and close the paddles 120, 122 of the anchor portion 106 and/or anchors 108.

In some implementations, the anchor portion 106 and/or anchors 108 can include attachment portions or gripping members. The illustrated gripping members can comprise clasps 130 that include a base or fixed arm 132, a moveable arm 134, optional friction-enhancing elements, other securing structures 136 (e.g., barbs, protrusions, ridges, grooves, textured surfaces, adhesive, etc.), and a joint portion 138. The fixed arms 132 are attached to the inner paddles 122. In some implementations, the fixed arms 132 are attached to the inner paddles 122 with the joint portion 138 disposed proximate the coaptation element 110. The joint portion 138 provides a spring force between the fixed and moveable arms 132, 134 of the clasp 130. The joint portion 138 can be any suitable joint, such as a flexible joint, a spring joint, a pivot joint, or the like. In some implementations, the joint portion 138 is a flexible piece of material integrally formed with the fixed and moveable arms 132, 134. The fixed arms 132 are attached to the inner paddles 122 and remain stationary or substantially stationary relative to the inner paddles 122 when the moveable arms 134 are opened to open the clasps 130 and expose the optional barbs or other optional friction-enhancing elements 136.

In some implementations, the clasps 130 are opened by applying tension to actuation lines 116 attached to the moveable arms 134, thereby causing the moveable arms 134 to articulate, flex, or pivot on the joint portions 138. The actuation lines 116 extend through the delivery system 102 (e.g., through a steerable catheter and/or an implant catheter). Other actuation mechanisms are also possible.

The actuation line 116 can take a wide variety of forms, such as, for example, a line, a suture, a wire, a rod, a catheter, or the like. The clasps 130 can be spring loaded so that in the closed position the clasps 130 continue to provide a pinching force on the grasped native leaflet. Optional barbs or other friction-enhancing elements 136 of the clasps 130 can grab, pinch, and/or pierce the native leaflets to further secure the native leaflets.

During implantation, the paddles 120, 122 can be opened and closed, for example, to grasp the native leaflets (e.g., native mitral valve leaflets, etc.) between the paddles 120, 122 and/or between the paddles 120, 122 and a coaptation element 110 (e.g., a spacer, plug, membrane, etc.). The clasps 130 can be used to grasp and/or further secure the native leaflets by engaging the leaflets with optional barbs or other optional friction-enhancing elements 136 and pinching the leaflets between the moveable and fixed arms 134, 132. The barbs or other friction-enhancing elements 136 (e.g., protrusions, ridges, grooves, textured surfaces, adhesive, etc.) of the clasps 130 increase friction with the leaflets or can partially or completely puncture the leaflets. The actuation lines 116 can be actuated separately so that each clasp 130 can be opened and closed separately. Separate operation allows one leaflet to be grasped at a time, or for the repositioning of a clasp 130 on a leaflet that was insufficiently grasped, without altering a successful grasp on the other leaflet. The clasps 130 can be opened and closed relative to the position of the inner paddle 122 (as long as the inner paddle is in an open or at least partially open position), thereby allowing leaflets to be grasped in a variety of positions as the particular situation requires.

Referring now to FIG. 8, the device 100 is shown in an elongated or fully open condition for deployment from an implant delivery catheter of the delivery system 102. The device 100 is disposed at the end of the catheter of the delivery system 102 in the fully open position. In the elongated condition the cap 114 is spaced apart from the coaptation element 110 such that the paddles 120, 122 are fully extended. In some implementations, an angle formed between the interior of the outer and inner paddles 120, 122 is approximately 180 degrees. The clasps 130 can be kept in a closed condition during deployment through the delivery system. The actuation lines 116 can extend and attach to the moveable arms 134.

Referring now to FIG. 9, the device 100 is shown in an elongated condition, similar to FIG. 8, but with the clasps 130 in a fully open position, ranging from about 140 degrees to about 200 degrees, from about 170 degrees to about 190 degrees, or about 180 degrees between fixed and moveable portions 132, 134 of the clasps 130.

Referring now to FIG. 10, the device 100 is shown in a shortened or fully closed condition. To move the device 100 from the elongated condition to the shortened condition, the actuation element 112 is retracted to pull the cap 114 towards the coaptation element 110. The connection portion(s) 126 (e.g., joint(s), flexible connection(s), etc.) between the outer paddle 120 and inner paddle 122 are constrained in movement such that compression forces acting on the outer paddle 120 from the cap 114 being retracted towards the coaptation element 110 cause the paddles or gripping elements to move radially outward. During movement from the open position to the closed position, the outer paddles 120 maintain an acute angle with the actuation element 112. The outer paddles 120 can optionally be biased toward a closed position. The inner paddles 122 during the same motion move through a considerably larger angle as they are oriented away from the coaptation element 110 in the open condition and collapse along the sides of the coaptation element 110 in the closed condition.

Referring now to FIGS. 11-13, the device 100 is shown in a partially open, grasp-ready condition. To transition from the fully closed to the partially open condition, the actuation element (e.g., actuation wire, actuation shaft, etc.) is extended to push the cap 114 away from the coaptation element 110, thereby pulling on the outer paddles 120, which in turn pull on the inner paddles 122, causing the anchors or anchor portion 106 to partially unfold. The actuation lines 116 are also retracted to open the clasps 130 so that the leaflets can be grasped. In some implementations, the pair of inner and outer paddles 122, 120 are moved in unison, rather than independently, by a single actuation element 112. Also, the positions of the clasps 130 are dependent on the positions of the paddles 122, 120. For example, referring to FIG. 10 closing the paddles 122, 120 also closes the clasps. In some implementations, the paddles 120, 122 can be independently controllable. In the example illustrated by FIG. 15, the device 100 can have two actuation elements 111, 113 and two independent caps 115, 117 (or other attachment portions), such that one independent actuation element (e.g., wire, shaft, etc.) and cap (or other attachment portion) are used to control one paddle, and the other independent actuation element and cap (or other attachment portion) are used to control the other paddle.

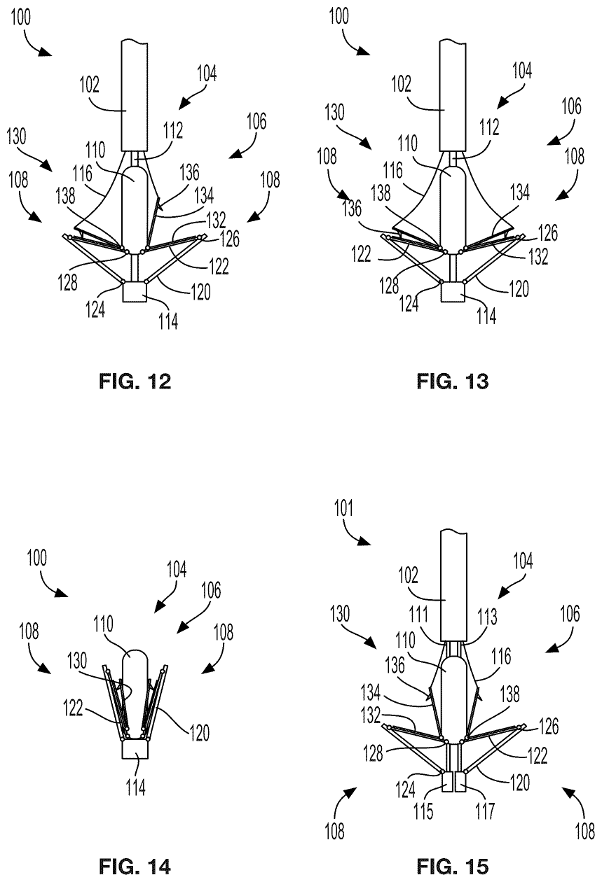

Referring now to FIG. 12, one of the actuation lines 116 is extended to allow one of the clasps 130 to close. Referring now to FIG. 13, the other actuation line 116 is extended to allow the other clasp 130 to close. Either or both of the actuation lines 116 can be repeatedly actuated to repeatedly open and close the clasps 130.

Referring now to FIG. 14, the device 100 is shown in a fully closed and deployed condition. The delivery system 102 and actuation element 112 are retracted and the paddles 120, 122 and clasps 130 remain in a fully closed position. Once deployed, the device 100 can be maintained in the fully closed position with a mechanical latch or can be biased to remain closed through the use of spring materials, such as steel, other metals, plastics, composites, etc. or shape-memory alloys such as Nitinol. For example, the connection portions 124, 126, 128, the joint portions 138, and/or the inner and outer paddles 122, and/or an additional biasing component (not shown) can be formed of metals such as steel or shape-memory alloy, such as Nitinol—produced in a wire, sheet, tubing, or laser sintered powder—and are biased to hold the outer paddles 120 closed around the coaptation element 110 and the clasps 130 pinched around native leaflets. Similarly, the fixed and moveable arms 132, 134 of the clasps 130 are biased to pinch the leaflets. In some implementations, the attachment or connection portions 124, 126, 128, joint portions 138, and/or the inner and outer paddles 122, and/or an additional biasing component (not shown) can be formed of any other suitably elastic material, such as a metal or polymer material, to maintain the device 100 in the closed condition after implantation.

FIG. 15 illustrates an example where the paddles 120, 122 are independently controllable. The device 101 illustrated by FIG. 15 is similar to the device illustrated by FIG. 11, except the device 101 of FIG. 15 includes an actuation element that is configured as two independent actuation elements 111, 113 that are coupled to two independent caps 115, 117. To transition a first inner paddle 122 and a first outer paddle 120 from the fully closed to the partially open condition, the actuation element 111 is extended to push the cap 115 away from the coaptation element 110, thereby pulling on the outer paddle 120, which in turn pulls on the inner paddle 122, causing the first anchor 108 to partially unfold. To transition a second inner paddle 122 and a second outer paddle 120 from the fully closed to the partially open condition, the actuation element 113 is extended to push the cap 115 away from the spacer or coaptation element 110, thereby pulling on the outer paddle 120, which in turn pulls on the inner paddle 122, causing the second anchor 108 to partially unfold. The independent paddle control illustrated by FIG. 15 can be implemented on any of the devices disclosed by the present application. For comparison, in the example illustrated by FIG. 11, the pair of inner and outer paddles 122, 120 are moved in unison, rather than independently, by a single actuation element 112.

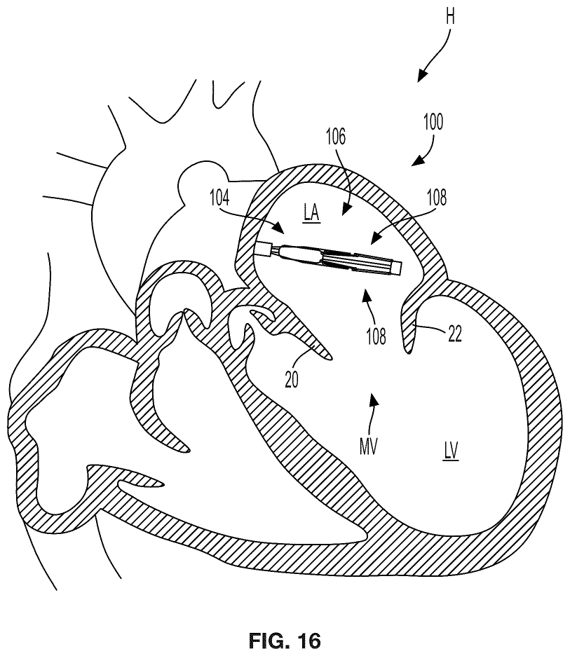

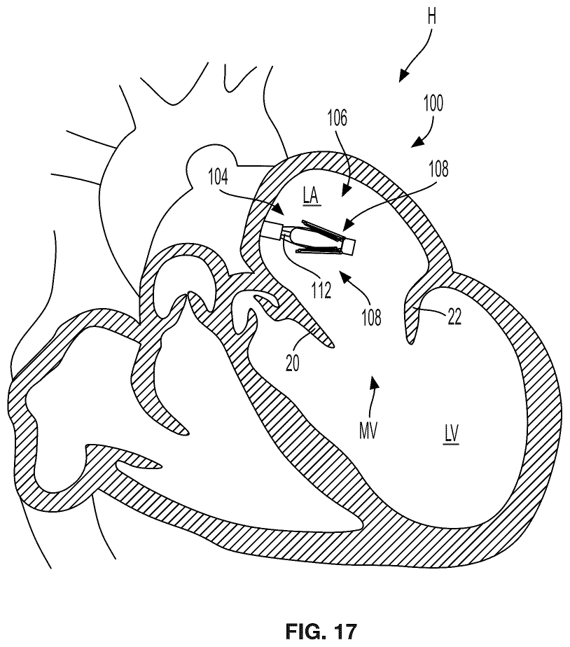

Referring now to FIGS. 16-21, the implantable device 100 of FIGS. 8-14 is shown being delivered and implanted within the native mitral valve MV of the heart H. Referring to FIG. 16, a delivery sheath/catheter is inserted into the left atrium LA through the septum and the implant/device 100 is deployed from the delivery catheter/sheath in the fully open condition as illustrated in FIG. 16. The actuation element 112 is then retracted to move the implant/device into the fully closed condition shown in FIG. 17.

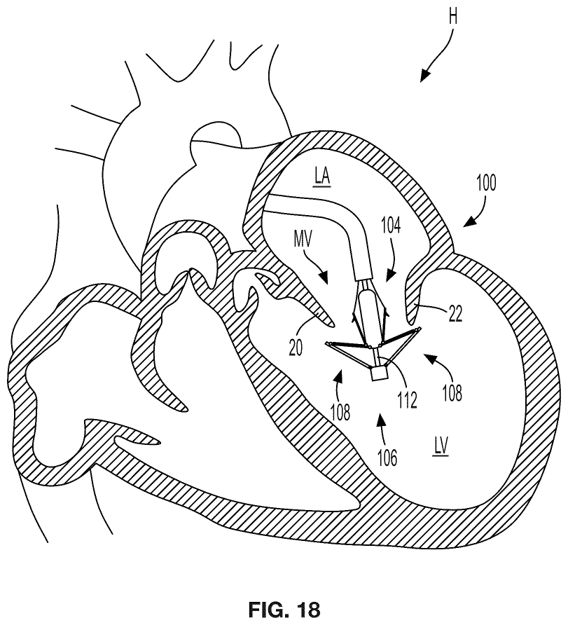

As can be seen in FIG. 18, the implant/device is moved into position within the mitral valve MV into the ventricle LV and partially opened so that the leaflets 20, 22 can be grasped. For example, a steerable catheter can be advanced and steered or flexed to position the steerable catheter as illustrated by FIG. 18. The implant catheter connected to the implant/device can be advanced from inside the steerable catheter to position the implant as illustrated by FIG. 18.

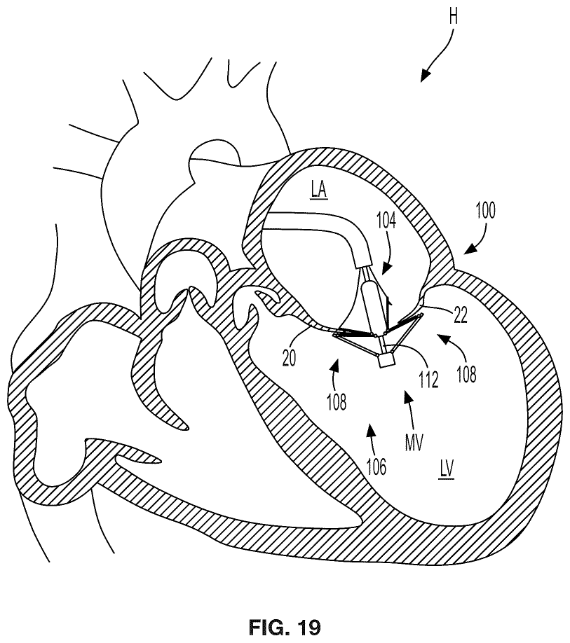

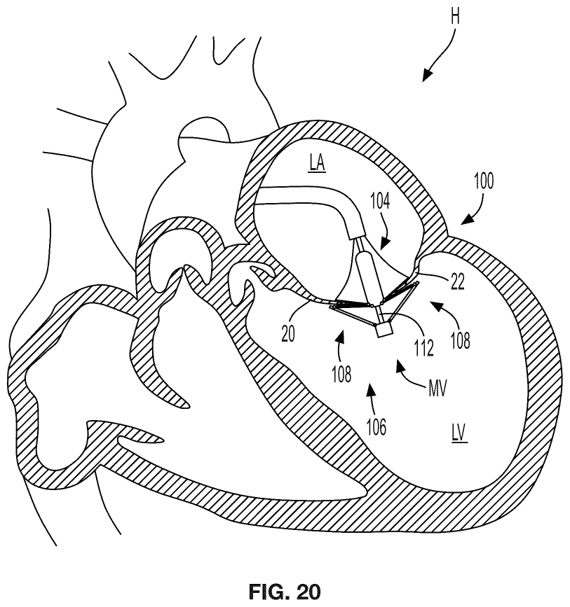

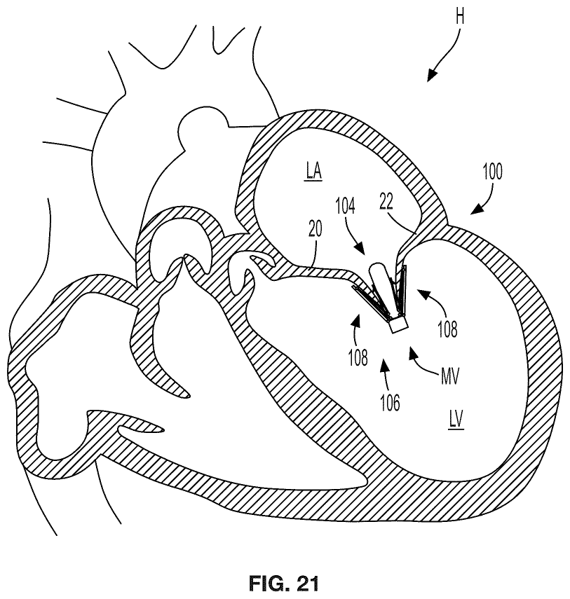

Referring now to FIG. 19, the implant catheter can be retracted into the steerable catheter to position the mitral valve leaflets 20, 22 in the clasps 130. An actuation line 116 is extended to close one of the clasps 130, capturing a leaflet 20. FIG. 20 shows the other actuation line 116 being then extended to close the other clasp 130, capturing the remaining leaflet 22. Lastly, as can be seen in FIG. 21, the delivery system 102 (e.g., steerable catheter, implant catheter, etc.), actuation element 112 and actuation lines 116 are then retracted and the device or implant 100 is fully closed and deployed in the native mitral valve MV.

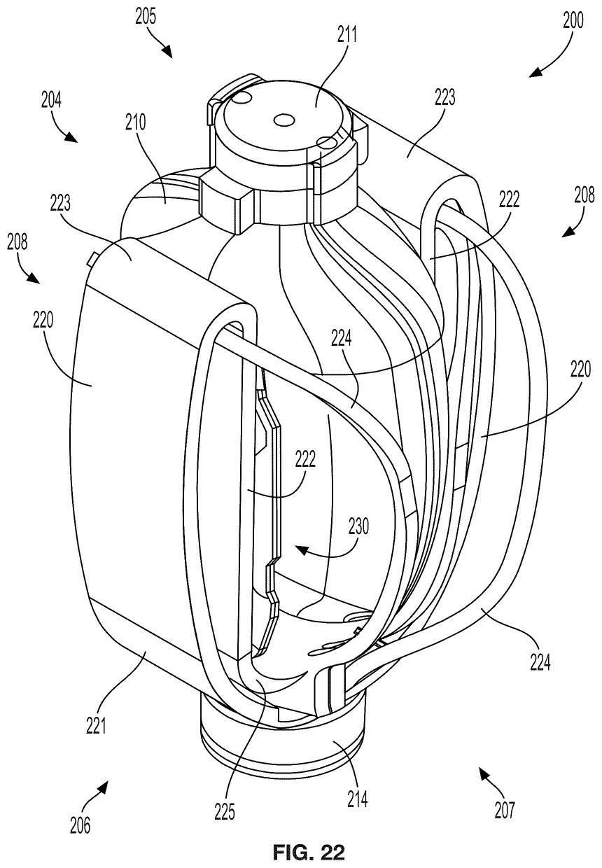

Any of the features disclosed by the present application can be used in a wide variety of different valve repair devices. FIGS. 22-24 illustrate examples of valve repair devices that can be modified to include any of the features disclosed by the present application. Any combination or sub-combination of the features disclosed by the present application can be combined with, substituted for, and/or added to any combination or sub-combination of the features of the valve repair devices illustrated by FIGS. 8-24.

Referring now to FIG. 22, an example of an implantable device or implant 200 is shown. The implantable device 200 is one of the many different configurations that the device 100 that is schematically illustrated in FIGS. 8-14 can take. The device 200 can include any other features for an implantable device or implant discussed in the present application, and the device 200 can be positioned to engage valve tissue 20, 22 as part of any suitable valve repair system (e.g., any valve repair system disclosed in the present application). The device/implant 200 can be a prosthetic spacer device, valve repair device, or another type of implant that attaches to leaflets of a native valve.

In some implementations, the implantable device or implant 200 includes a coaptation portion 204, a proximal or attachment portion 205, an anchor portion 206, and a distal portion 207. In some implementations, the coaptation portion 204 of the device optionally includes a coaptation element 210 (e.g., a spacer, coaption element, plug, membrane, sheet, gap filler, etc.) 210 for implantation between leaflets of a native valve. In some implementations, the anchor portion 206 includes a plurality of anchors 208. The anchors can be configured in a variety of ways. In some implementations, each anchor 208 includes outer paddles 220, inner paddles 222, paddle extension members or paddle frames 224, and clasps 230. In some implementations, the attachment portion 205 includes a first or proximal collar 211 (or other attachment element) for engaging with a capture mechanism of a delivery system. A delivery system for the device 200 can be the same as or similar to delivery system 102 described above and can comprise one or more of a catheter, a sheath, a guide catheter/sheath, a delivery catheter/sheath, a steerable catheter, an implant catheter, a tube, a channel, a pathway, combinations of these, etc.

In some implementations, the coaptation element 210 and paddles 220, 222 are formed from a flexible material that can be a metal fabric, such as a mesh, woven, braided, or formed in any other suitable way or a laser cut or otherwise cut flexible material. The material can be cloth, shape-memory alloy wire—such as Nitinol—to provide shape-setting capability, or any other flexible material suitable for implantation in the human body.

An actuation element (e.g., actuation shaft, actuation rod, actuation tube, actuation wire, actuation line, etc.) can extend from a delivery system (not shown) to engage and enable actuation of the implantable device or implant 200. In some implementations, the actuation element extends through the proximal collar 211, and spacer or coaptation element 210 to engage a cap 214 of the distal portion 207. The actuation element can be configured to removably engage the cap 214 with a threaded connection, or the like, so that the actuation element can be disengaged and removed from the device 200 after implantation.

The coaptation element 210 extends from the proximal collar 211 (or other attachment element) to the inner paddles 222. In some implementations, the coaptation element 210 has a generally elongated and round shape, though other shapes and configurations are possible. In some implementations, the coaptation element 210 has an elliptical shape or cross-section when viewed from above and has a tapered shape or cross-section when seen from a front view and a round shape or cross-section when seen from a side view. A blend of these three geometries can result in the three-dimensional shape of the illustrated coaptation element 210 that achieves the benefits described herein. The round shape of the coaptation element 210 can also be seen, when viewed from above, to substantially follow or be close to the shape of the paddle frames 224.

The size and/or shape of the coaptation element 210 can be selected to minimize the number of implants that a single patient will require (preferably one), while at the same time maintaining low transvalvular gradients. In some implementations, the anterior-posterior distance at the top of the coaptation element is about 5 mm, and the medial-lateral distance of the coaptation element at its widest is about 10 mm. In some implementations, the overall geometry of the device 200 can be based on these two dimensions and the overall shape strategy described above. It should be readily apparent that the use of other anterior-posterior distance anterior-posterior distance and medial-lateral distance as starting points for the device will result in a device having different dimensions. Further, using other dimensions and the shape strategy described above will also result in a device having different dimensions.

In some implementations, the outer paddles 220 are jointably attached to the cap 214 of the distal portion 207 by connection portions 221 and to the inner paddles 222 by connection portions 223. The inner paddles 222 are jointably attached to the coaptation element by connection portions 225. In this manner, the anchors 208 are configured similar to legs in that the inner paddles 222 are like upper portions of the legs, the outer paddles 220 are like lower portions of the legs, and the connection portions 223 are like knee portions of the legs.

In some implementations, the inner paddles 222 are stiff, relatively stiff, rigid, have rigid portions and/or are stiffened by a stiffening member (e.g., bar, strut, etc.) or a fixed portion of the clasps 230. The inner paddle 222, the outer paddle 220, and the coaptation element can all be interconnected as described herein.

In some implementations, the paddle frames 224 are attached to the cap 214 at the distal portion 207 and extend to the connection portions 223 between the inner and outer paddles 222, 220. In some implementations, the paddle frames 224 are formed of a material that is more rigid and stiff than the material forming the paddles 222, 220 so that the paddle frames 224 provide support for the paddles 222, 220.

The paddle frames 224 can provide additional pinching force between the inner paddles 222 and the coaptation element 210 and assist in wrapping the leaflets around the sides of the coaptation element 210. That is, the paddle frames 224 can be configured with a round three-dimensional shape extending from the cap 214 to the connection portions 223 of the anchors 208. The connections between the paddle frames 224, the outer and inner paddles 220, 222, the cap 214, and the coaptation element 210 can constrain each of these parts to the movements and positions described herein. In particular the connection portion 223 is constrained by its connection between the outer and inner paddles 220, 222 and by its connection to the paddle frame 224. Similarly, the paddle frame 224 is constrained by its attachment to the connection portion 223 (and thus the inner and outer paddles 222, 220) and to the cap 214.

The wide configuration of the paddle frames 224 provides increased surface area compared to the inner paddles 222 alone. The increased surface area can distribute the clamping force of the paddles 220 and paddle frames 224 against the native leaflets over a relatively larger surface of the native leaflets in order to further protect the native leaflet tissue.

Additional features of the device 200, modified versions of the device, delivery systems for the device, and methods for using the device and delivery system are disclosed by Patent Cooperation Treaty International Application No. PCT/US2018/028189 (International Publication No. WO 2018/195215). Any combination or sub-combination of the features disclosed by the present application can be combined with any combination or sub-combination of the features disclosed by Patent Cooperation Treaty International Application No. PCT/US2018/028189 (International Publication No. WO 2018/195215). Patent Cooperation Treaty International Application No. PCT/US2018/028189 (International Publication No. WO 2018/195215) is incorporated herein by reference in its entirety.

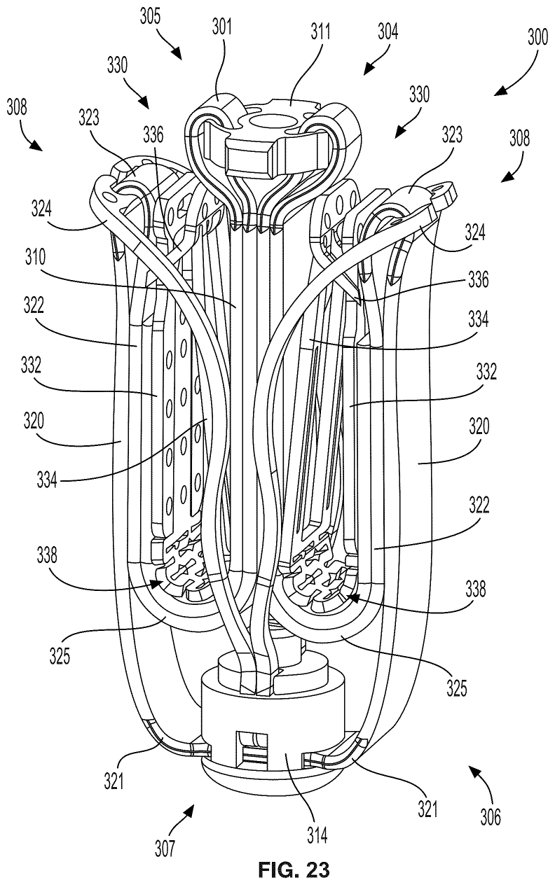

Referring now to FIG. 23, an example of an implantable device or implant 300 is shown. The implantable device 300 is one of the many different configurations that the device 100 that is schematically illustrated in FIGS. 8-14 can take. The device 300 can include any other features for an implantable device or implant discussed in the present application, and the device 300 can be positioned to engage valve tissue 20, 22 as part of any suitable valve repair system (e.g., any valve repair system disclosed in the present application).

The implantable device or implant 300 includes a proximal or attachment portion 305, an anchor portion 306, and a distal portion 307. In some implementations, the device/implant 300 includes a coaptation portion 304, and the coaptation portion 304 can optionally include a coaptation element 310 (e.g., spacer, plug, membrane, sheet, etc.) for implantation between the leaflets 20, 22 of the native valve. In some implementations, the anchor portion 306 includes a plurality of anchors 308. In some implementations, each anchor 308 can include one or more paddles, e.g., outer paddles 320, inner paddles 322, paddle extension members (e.g., leaf spring, shaped wire, etc.) or paddle frames 324. The anchors can also include and/or be coupled to clasps 330. In some implementations, the attachment portion 305 includes a first or proximal collar 311 (or other attachment element) for engaging with a capture mechanism of a delivery system.

The anchors 308 can be attached to the other portions of the device and/or to each other in a variety of different ways (e.g., directly, indirectly, welding, sutures, adhesive, links, latches, integrally formed, a combination of some or all of these, etc.). In some implementations, the anchors 308 are attached to a coaptation element 310 by connection portions 325 and to a cap 314 by connection portions 321.

The anchors 308 can comprise first portions or outer paddles 320 and second portions or inner paddles 322 separated by connection portions 323. The connection portions 323 can be attached to paddle frames 324 that are hingeably attached to a cap 314 or other attachment portion. In this manner, the anchors 308 are configured similar to legs in that the inner paddles 322 are like upper portions of the legs, the outer paddles 320 are like lower portions of the legs, and the connection portions 323 are like knee portions of the legs.

In some implementations with a coaptation element 310, the coaptation element 310 and the anchors 308 can be coupled together in various ways. As shown in the illustrated example, the coaptation element 310 and the anchors 308 can be coupled together by integrally forming the coaptation element 310 and the anchors 308 as a single, unitary component. This can be accomplished, for example, by forming the coaptation element 310 and the anchors 308 from a continuous strip 301 of a braided or woven material, such as braided or woven nitinol wire. In the illustrated example, the coaptation element 310, the outer paddle portions 320, the inner paddle portions 322, and the connection portions 321, 323, 325 are formed from a continuous strip of fabric 301.

Like the anchors 208 of the implantable device or implant 200 described above, the anchors 308 can be configured to move between various configurations by axially moving the distal end of the device (e.g., cap 314, etc.) relative to the proximal end of the device (e.g., proximal collar 311 or other attachment element, etc.). This movement can be along a longitudinal axis extending between the distal end (e.g., cap 314, etc.) and the proximal end (e.g., collar 311 or other attachment element, etc.) of the device.

In some implementations, in the straight configuration, the paddle portions 320, 322 are aligned or straight in the direction of the longitudinal axis of the device. In some implementations, the connection portions 323 of the anchors 308 are adjacent the longitudinal axis of the spacer or coaptation element 310. From the straight configuration, the anchors 308 can be moved to a fully folded configuration (e.g., FIG. 23), e.g., by moving the proximal end and distal end toward each other and/or toward a midpoint or center of the device.

In some implementations, the clasps comprise a moveable arm coupled to an anchor. In some implementations, the clasps 330 include a base or fixed arm 332, a moveable arm 334, optional barbs/friction-enhancing elements 336, and a joint portion 338. The fixed arms 332 are attached to the inner paddles 322, with the joint portion 338 disposed proximate the coaptation element 310. The joint portion 338 is spring-loaded so that the fixed and moveable arms 332, 334 are biased toward each other when the clasp 330 is in a closed condition.

The fixed arms 332 are attached to the inner paddles 322 through holes or slots with sutures. The fixed arms 332 can be attached to the inner paddles 322 with any suitable means, such as screws or other fasteners, crimped sleeves, mechanical latches or snaps, welding, adhesive, or the like. The fixed arms 332 remain substantially stationary relative to the inner paddles 322 when the moveable arms 334 are opened to open the clasps 330 and expose the optional barbs 336. The clasps 330 are opened by applying tension to actuation lines attached to the moveable arms 334, thereby causing the moveable arms 334 to articulate, pivot, and/or flex on the joint portions 338.

In short, the implantable device or implant 300 is similar in configuration and operation to the implantable device or implant 200 described above, except that the coaptation element 310, outer paddles 320, inner paddles 322, and connection portions 321, 323, 325 are formed from the single strip of material 301. In some implementations, the strip of material 301 is attached to the proximal collar 311, cap 314, and paddle frames 324 by being woven or inserted through openings in the proximal collar 311, cap 314, and paddle frames 324 that are configured to receive the continuous strip of material 301. The continuous strip 301 can be a single layer of material or can include two or more layers. In some implementations, portions of the device 300 have a single layer of the strip of material 301 and other portions are formed from multiple overlapping or overlying layers of the strip of material 301.

For example, FIG. 23 shows a coaptation element 310 and inner paddles 322 formed from multiple overlapping layers of the strip of material 301. The single continuous strip of material 301 can start and end in various locations of the device 300. The ends of the strip of material 301 can be in the same location or different locations of the device 300. For example, in the illustrated example of FIG. 23, the strip of material 301 begins and ends in the location of the inner paddles 322.

As with the implantable device or implant 200 described above, the size of the coaptation element 310 can be selected to minimize the number of implants that a single patient will require (preferably one), while at the same time maintaining low transvalvular gradients. In particular, forming many components of the device 300 from the strip of material 301 allows the device 300 to be made smaller than the device 200. For example, in some implementations, the anterior-posterior distance at the top of the coaptation element 310 is less than 2 mm, and the medial-lateral distance of the device 300 (i.e., the width of the paddle frames 324 which are wider than the coaptation element 310) at its widest is about 5 mm.

Additional features of the device 300, modified versions of the device, delivery systems for the device, and methods for using the device and delivery system are disclosed by Patent Cooperation Treaty International Application No. PCT/US2019/055320 (International Publication No. WO 2020/076898). Any combination or sub-combination of the features disclosed by the present application can be combined with any combination or sub-combination of the features disclosed by Patent Cooperation Treaty International Application No. PCT/US2019/055320 (International Publication No. WO 2020/076898). Patent Cooperation Treaty International Application No. PCT/US2019/055320 (International Publication No. WO 2020/076898) is incorporated herein by reference in its entirety.

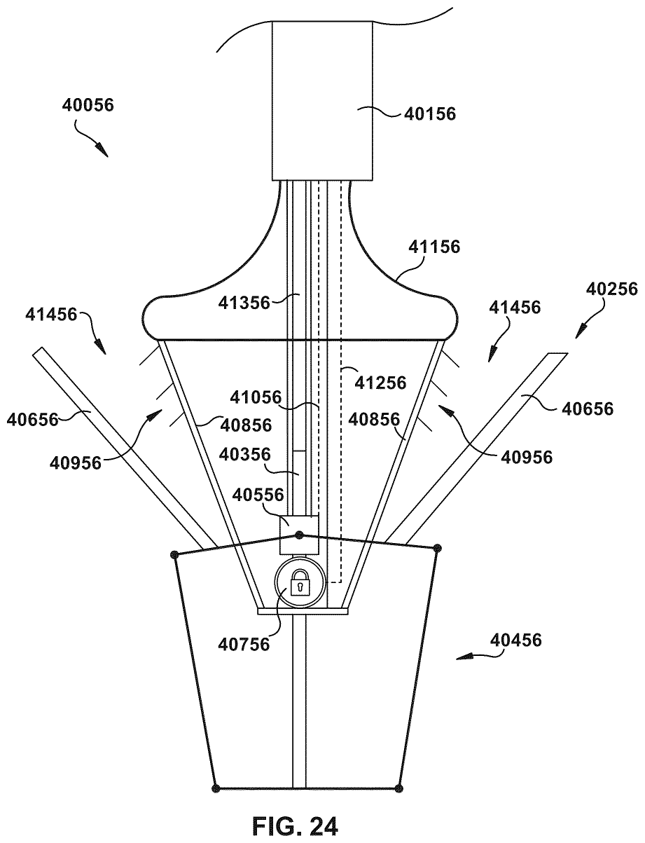

FIG. 24 illustrates another example of one of the many valve repair systems 40056 for repairing a native valve of a patient that the concepts of the present application can be applied to. The valve repair system 40056 includes a delivery device 40156 and a valve repair device 40256.

The valve repair device 40256 includes a base assembly 40456, a pair of paddles 40656, and a pair of gripping members 40856 (e.g., clasp, clip, arm, etc.). In one example, the paddles 40656 can be integrally formed with the base assembly. For example, the paddles 40656 can be formed as extensions of links of the base assembly. In the illustrated example, the base assembly 40456 of the valve repair device 40256 has a shaft 40356, a coupler 40556 configured to move along the shaft, and a lock 40756 configured to lock the coupler in a stationary position on the shaft. The coupler 40556 is mechanically connected to the paddles 40656, such that movement of the coupler 40556 along the shaft 40356 causes the paddles to move between an open position and a closed position. In this way, the coupler 40556 serves as a means for mechanically coupling the paddles 40656 to the shaft 40356 and, when moving along the shaft 40356, for causing the paddles 40656 to move between their open and closed positions.

In some implementations, the gripping members 40856 are pivotally connected to the base assembly 40456 (e.g., the gripping members 40856 can be pivotally connected to the shaft 40356, or any other suitable member of the base assembly), such that the gripping members can be moved to adjust the width of the opening 41456 between the paddles 40656 and the gripping members 40856. The gripping member 40856 can include an optionally barbed portion 40956 for attaching the gripping members to valve tissue when the valve repair device 40256 is attached to the valve tissue. When the paddles 40656 are in the closed position, the paddles engage the gripping members 40856, such that, when valve tissue is attached to the barbed portion 40956 of the gripping members, the paddles secure the valve repair device 40256 to the valve tissue. In some implementations, the gripping members 40856 are configured to engage the paddles 40656 such that the barbed portion 40956 engages the valve tissue and the paddles 40656 to secure the valve repair device 40256 to the valve tissue. For example, in certain situations, it can be advantageous to have the paddles 40656 maintain an open position and have the gripping members 40856 move outward toward the paddles 40656 to engage valve tissue and the paddles 40656.

While the example shown in FIG. 24 illustrates a pair of paddles 40656 and a pair of gripping members 40856, it should be understood that the valve repair device 40256 can include any suitable number of paddles and gripping members.

In some implementations, the valve repair system 40056 includes a placement shaft 41356 that is removably attached to the shaft 40356 of the base assembly 40456 of the valve repair device 40256. After the valve repair device 40256 is secured to valve tissue, the placement shaft 41356 is removed from the shaft 40356 to remove the valve repair device 40256 from the remainder of the valve repair system 40056, such that the valve repair device 40256 can remain attached to the valve tissue, and the delivery device 40156 can be removed from a patient's body.

The valve repair system 40056 can also include a paddle control mechanism 41056 (e.g., relatively movable tube(s), shaft(s), etc.), a gripper control mechanism 41156 (e.g., wire(s), line(s), suture(s), etc.), and a lock control mechanism 41256 (e.g., relatively movable tube(s), shaft(s), wire(s), line(s), suture(s), etc.). The paddle control mechanism 41056 is mechanically attached to the coupler 40556 to move the coupler along the shaft, which causes the paddles 40656 to move between the open and closed positions. The paddle control mechanism 41056 can take any suitable form, such as, for example, a shaft or rod. For example, the paddle control mechanism can comprise a hollow shaft, a catheter tube or a sleeve that fits over the placement shaft 41356 and the shaft 40356 and is connected to the coupler 40556.

The gripper control mechanism 41156 is configured to move the gripping members 40856 such that the width of the opening 41456 between the gripping members and the paddles 40656 can be altered. The gripper control mechanism 41156 can take any suitable form, such as, for example, a line, a suture or wire, a rod, a catheter, etc.

The lock control mechanism 41256 is configured to lock and unlock the lock. The lock 40756 locks the coupler 40556 in a stationary position with respect to the shaft 40356 and can take a wide variety of different forms and the type of lock control mechanism 41256 can be dictated by the type of lock used. In examples in which the lock 40756 includes a pivotable plate, the lock control mechanism 41256 is configured to engage the pivotable plate to move the plate between the tilted and substantially non-tilted positions. The lock control mechanism 41256 can be, for example, a rod, a suture, a wire, or any other member that is capable of moving a pivotable plate of the lock 40756 between a tilted and substantially non-tilted position.

The valve repair device 40256 is movable from an open position to a closed position. The base assembly 40456 includes links that are moved by the coupler 40556. The coupler 40556 is movably attached to the shaft 40356. In order to move the valve repair device from the open position to the closed position, the coupler 40556 is moved along the shaft 40356, which moves the links.

The gripper control mechanism 41156 is moves the gripping members 40856 to provide a wider or a narrower gap at the opening 41456 between the gripping members and the paddles 40656. In the illustrated example, the gripper control mechanism 41156 includes a line, such as a suture, a wire, etc. that is connected to an opening in an end of the gripper members 40856. When the line(s) is pulled, the gripping members 40856 move inward, which causes the opening 41456 between the gripping members and the paddles 40656 to become wider.

In order to move the valve repair device 40256 from the open position to the closed position, the lock 40756 is moved to an unlocked condition by the lock control mechanism 41256. Once the lock 40756 is in the unlocked condition, the coupler 40556 can be moved along the shaft 40356 by the paddle control mechanism 41056.

After the paddles 40656 are moved to the closed position, the lock 40756 is moved to the locked condition by the lock control mechanism 41256 to maintain the valve repair device 40256 in the closed position. After the valve repair device 40256 is maintained in the locked condition by the lock 40756, the valve repair device 40256 is removed from the delivery device 40156 by disconnecting the shaft 40356 from the placement shaft 41356. In addition, the valve repair device 40256 is disengaged from the paddle control mechanism 41056, the gripper control mechanism 41156, and the lock control mechanism 41256.

Additional features of the device 40256, modified versions of the device, delivery systems for the device, and methods for using the device and delivery system are disclosed by Patent Cooperation Treaty International Application No. PCT/US2019/012707 (International Publication No. WO 2019139904). Any combination or sub-combination of the features disclosed by the present application can be combined with any combination or sub-combination of the features disclosed by Patent Cooperation Treaty International Application No. PCT/US2019/012707 (International Publication No. WO 2019139904). Patent Cooperation Treaty International Application No. PCT/US2019/012707 (International Publication No. WO 2019139904) is incorporated herein by reference in its entirety.

Clasps or leaflet gripping devices disclosed herein can take a wide variety of different forms. Examples of clasps are disclosed by Patent Cooperation Treaty International Application No. PCT/US2018/028171 (International Publication No. WO 2018195201). Any combination or sub-combination of the features disclosed by the present application can be combined with any combination or sub-combination of the features disclosed by Patent Cooperation Treaty International Application No. PCT/US2018/028171 (International Publication No. WO 2018195201). Patent Cooperation Treaty International Application No. PCT/US2018/028171 (International Publication No. WO 2018195201) is incorporated herein by reference in its entirety.

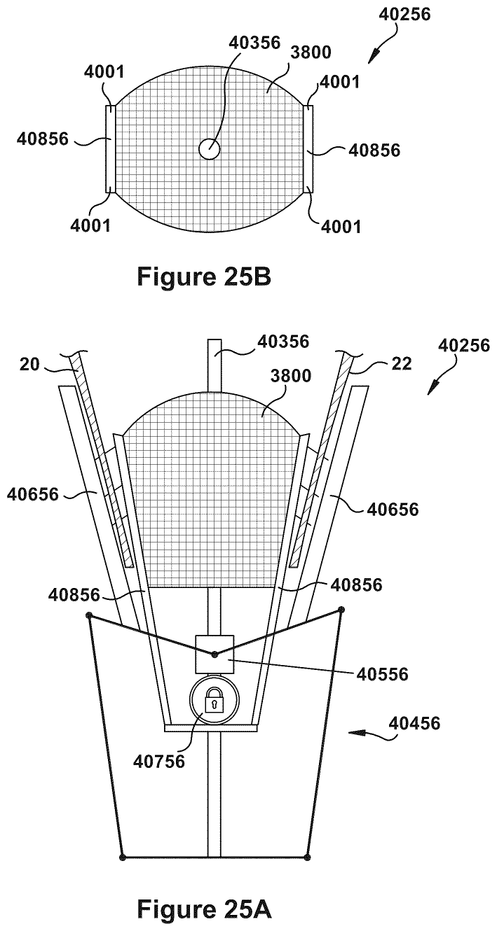

Referring to FIGS. 25A-25B, an example implementation of a valve repair device 40256 has a coaptation element 3800. The valve repair device 40256 can have the same configuration as the valve repair device illustrated by FIG. 24 with the addition of the coaptation element. The coaptation element 3800 can take a wide variety of different forms. The coaptation element 3800 can be compressible and/or expandable. For example, the coaptation element can be compressed to fit inside one or more catheters of a delivery system, can expand when moved out of the one or more catheters, and/or can be compressed by the paddles 40656 to adjust the size of the coaptation element. In the example illustrated by FIGS. 25A and 25B, the size of the coaptation element 3800 can be reduced by squeezing the coaptation element with the paddles 40656 and can be increased by moving the paddles 40656 away from one another. The coaptation element 3800 can extend past outer edges 4001 of the gripping members or clasps 40856 as illustrated for providing additional surface area for closing the gap of a mitral valve.

The coaptation element 3800 can be coupled to the valve repair device 40256 in a variety of different ways. For example, the coaptation element 3800 can be fixed to the shaft 40356, can be slidably disposed around the shaft, can be connected to the coupler 40556, can be connected to the lock 40755, and/or can be connected to a central portion of the clasps or gripping members 40856. In some implementations, the coupler 40556 can take the form of the coaptation element 3800. That is, a single element can be used as the coupler 40556 that causes the paddles 40656 to move between the open and closed positions and the coaptation element 3800 that closes the gap between the leaflets 20, 22 when the valve repair device 40256 is attached to the leaflets.

The coaptation element 3800 can be disposed around one or more of the shafts or other control elements of the valve repair system 40056. For example, the coaptation element 3800 can be disposed around the shaft 40356, the shaft 41356, the paddle control mechanism 41056, and/or the lock control mechanism 41256.

The valve repair device 40256 can include any other features for a valve repair device discussed in the present application, and the valve repair device 40256 can be positioned to engage valve tissue as part of any suitable valve repair system (e.g., any valve repair system disclosed in the present application). Additional features of the device 40256, modified versions of the device, delivery systems for the device, and methods for using the device and delivery system are disclosed by Patent Cooperation Treaty International Application No. PCT/US2019/012707 (International Publication No. WO 2019139904). Any combination or sub-combination of the features disclosed by the present application can be combined with any combination or sub-combination of the features disclosed by Patent Cooperation Treaty International Application No. PCT/US2019/012707 (International Publication No. WO 2019139904).

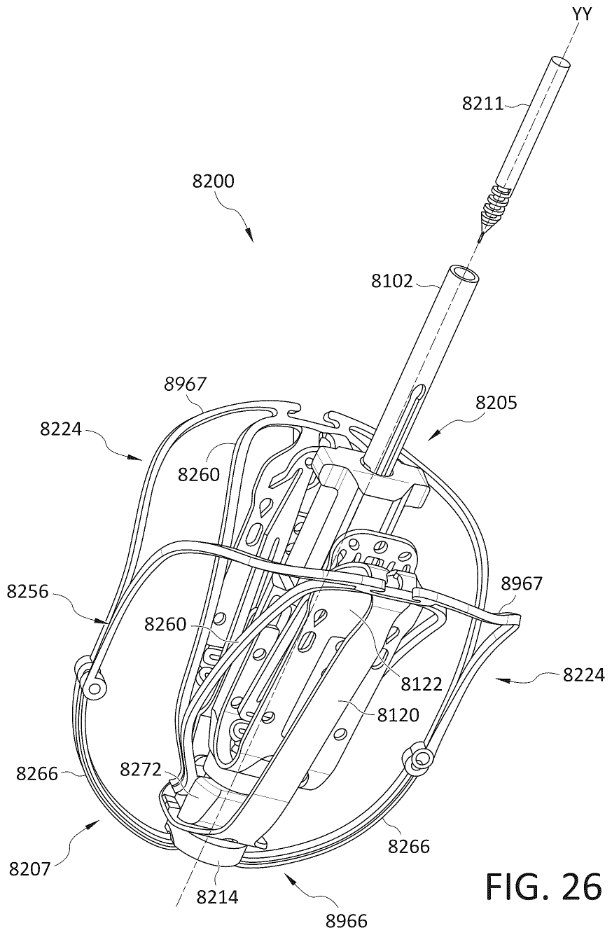

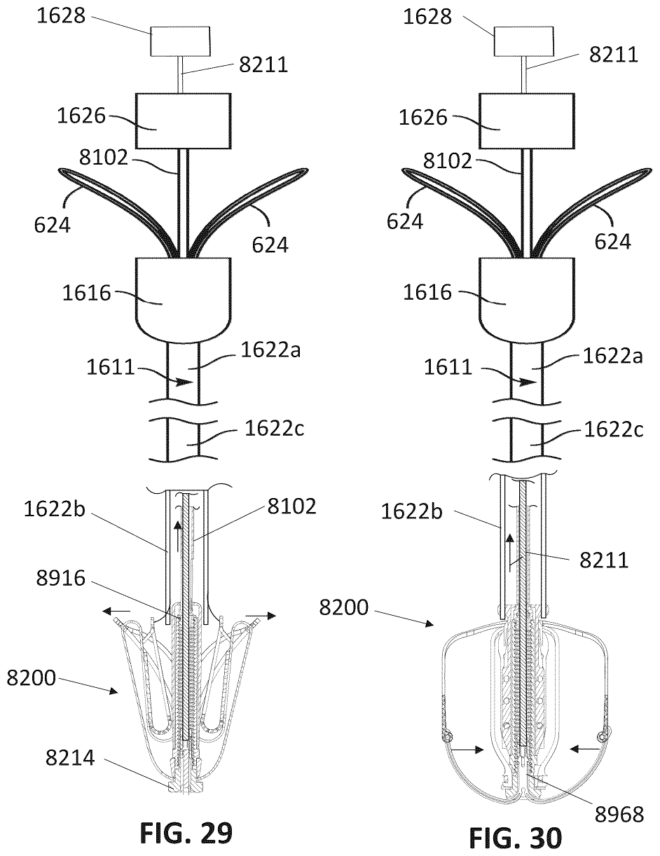

FIGS. 26-30 illustrate another example of one of the many valve repair systems for repairing a native valve of a patient that the concepts of the present application can be applied to. Referring to FIGS. 29 and 30, the valve repair system includes an implant catheter assembly 1611 and an implantable valve repair device 8200. Referring to FIGS. 26-28, the implantable device 8200 includes a proximal or attachment portion 8205, paddle frames 8224, and a distal portion 8207. The attachment portion 8205, the distal portion 8207, and the paddle frames 8224 can be configured in a variety of ways.

In the example illustrated in FIG. 26, the paddle frames 8224 can be symmetric along longitudinal axis YY. However, in some implementations, the paddle frames 8224 are not symmetric about the axis YY. Moreover, referring to FIG. 26, the paddle frames 8224 include outer or adjustable width frame portions 8256 and optional inner frame portions 8260.

In some implementations, a connector 8966 (e.g., shaped metal component, shaped plastic component, tether, wire, strut, line, cord, suture, etc.) attaches to the outer or adjustable width frame portions 8256 at outer ends or connection portions 8266 of the connector 8966 and to a coupler 8972 at an inner end 8968 of the connector 8966 (see FIG. 28). Between the connector 8966 and the attachment portion 8205, the outer or adjustable width frame portions 8256 form a curved shape. In the illustrated example, the shape of the outer or adjustable width frame portions 8256 resembles an apple shape in which the outer or adjustable width frame portions 8256 are wider toward the attachment portion 8205 and narrower toward the distal portion 8207. In some implementations, however, the outer frame portions 8256 can be otherwise shaped.

In the example illustrated in FIG. 26, the outer or adjustable width frame members 8256 (e.g., wire frames, metal frames, etc.) comprise anterior and posterior frame members 8967. The anterior and posterior frame members 8967 are connected to connection portions 8266 of the connector 8966. In the illustrated example, the connector 8966 is attached to both of the adjustable width frame members 8967 via two joints, such as suture joints. In some implementations, however, the connector 8966 can attach to both the anterior and posterior frame members 8967 in any suitable manner.

The inner frame portions 8260 extend from the attachment portion 8205 toward the distal portion 8207. The inner frame portions 8260 then extend inward to form retaining portions 8272 that are attached to the actuation cap 8214. The retaining portions 8272 and the actuation cap 8214 can be configured to attach in any suitable manner.

In some implementations, the optional inner frame portions 8260 are rigid frame portions, while the outer or adjustable width frame portions 8256 are flexible frame portions. The proximal end of the outer frame portions 8256 can connect to or be integrally formed with the proximal end of the inner frame portions 8260, as illustrated in FIG. 26.

The width adjustment element 8211 (e.g., width adjustment wire, width adjustment shaft, width adjustment tube, width adjustment line, width adjustment cord, width adjustment suture, width adjustment screw or bolt, etc.) is configured to move the outer frame portions 8256 from the expanded position to the narrowed position by pulling the inner end 8968 (FIG. 28) and portions of the connector 8266 into the actuation cap 8214. The actuation element 8102 is configured to move the inner frame portions 8260 to open and close the paddles in accordance with some implementations disclosed herein.

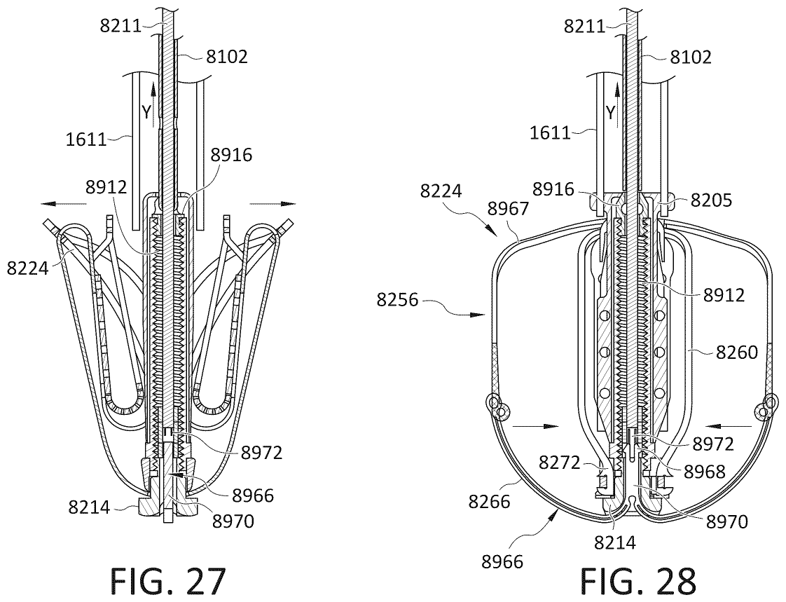

As shown in FIGS. 27 and 28, the connector 8966 has an inner end 8968 that engages with the width adjustment element 8211 such that a user can move the inner end 8968 inside the receiver 8912 (e.g., an internally threaded element, a column, a conduit, a hollow member, a notched receiving portion, a tube, a shaft, a sleeve, a post, a housing, a cylinder, tracks, etc.) to move the outer frame portions 8256 between a narrowed position and an expanded position. In the illustrated example, the inner end 8968 includes a post 8970 that attaches the outer portions 8266 of the connector 8966 to a coupler 8972. The coupler 8972 is configured to attach and detach from both the width adjustment element 8211 and the receiver 8912. The coupler 8972 can take a wide variety of different forms. For example, the coupler 8972 can include one or more of a threaded connection, features that mate with threads, detent connections, such as outwardly biased arms, walls, or other portions. When the coupler 8972 is attached to the width adjustment element 8211, the coupler is released from the receiver 8912. When the coupler 8972 is detached from the width adjustment element 8211, the coupler is secured to the receiver 8912. The inner end 8968 of the connector can, however, be configured in a variety of ways. Any configuration that can suitably attach the outer frame portions 8256 to the coupler to allow the width adjustment element 8211 to move the outer frame portions 8256 between the narrowed position and the expanded position can be used. The coupler can be configured in a variety of ways as well and can be a separate component or be integral with another portion of the device, e.g., of the connector or inner end of the connector.

The width adjustment element 8211 allows a user to expand or contract the outer frame portions 8256 of the implantable device 8200. In the example illustrated in FIGS. 27 and 28, the width adjustment element 8211 includes an externally threaded end that is threaded into the coupler 8972. The width adjustment element 8211 moves the coupler in the receiver 8912 to adjust the width of the outer frame portions 8256. When the width adjustment element 8211 is unscrewed from the coupler 8972, the coupler engages the inner surface of the receiver 8912 to set the width of the outer frame portions 8256.

In some implementations, the receiver 8912 can be integrally formed with a distal cap 8214. Moving the cap 8214 relative to a body of the attachment portion 8205 opens and closes the paddles. In the illustrated example, the receiver 8912 slides inside the body of the attachment portion. When the coupler 8972 is detached from the width adjustment element 8211, the width of the outer frame portions 8256 is fixed while the actuation element 8102 moves the receiver 8912 and cap 8214 relative to a body of the attachment portion 8205. Movement of the cap can open and close the device in the same manner as some implementations disclosed above.

In the illustrated example, a driver head 8916 is disposed at a proximal end of the actuation element 8102. The driver head 8916 releasably couples the actuation element 8102 to the receiver 8912. In the illustrated example, the width adjustment element 8211 extends through the actuation element 8102. The actuation element is axially advanced in the direction opposite to direction Y to move the distal cap 8214. Movement of the distal cap 8214 relative to the attachment portion 8205 is effective to open and close the paddles, as indicated by the arrows in FIG. 27. That is, movement of the distal cap 8214 in the direction Y closes the device and movement of the distal cap in the direction opposite to direction Y opens the device.

Also illustrated in FIGS. 27 and 28, the width adjustment element 8211 extends through the actuation element 8102, the driver head 8916, and the receiver 8912 to engage the coupler 8972 attached to the inner end 8968. The movement of the outer frame portions 8256 to the narrowed position can allow the device or implant 8200 to maneuver more easily into position for implantation in the heart by reducing the contact and/or friction between the native structures of the heart—e.g., chordae—and the device 8200. The movement of the outer frame portions 8256 to the expanded position provides the anchor portion of the device or implant 8200 with a larger surface area to engage and capture leaflet(s) of a native heart valve.

Referring to FIGS. 29 and 30, an example of an implant catheter assembly 1611 in which clasp actuation lines 624 extend through a handle 1616, the actuation element 8102 is coupled to a paddle actuation control 1626, and the width adjustment element 8211 is coupled to a paddle width control 1628. A proximal end portion 1622a of the shaft or catheter of the implant catheter assembly 1611 can be coupled to the handle 1616, and a distal end portion 1622b of the shaft or catheter can be coupled to the implantable device 8200. The actuation element 8102 can extend distally from the paddle actuation control 1626, through the handle 1616, through the delivery shaft or catheter of the implant catheter assembly 1611, and through the proximal end of the device 8200, where it couples with the driver head 8916. The actuation element 8102 can be axially movable relative to the outer shaft of the implant catheter assembly 1611 and the handle 1616 to open and close the device.

The width adjustment element 8211 can extend distally from the paddle width control 1628, through the paddle actuation control 1626 and through the actuation element 8102 (and, consequently, through the handle 1616, the outer shaft of the implant catheter assembly 1611, and through the device 8200), where it couples with the movable coupler 8972. The width adjustment element 8211 can be axially movable relative to the actuation element 8102, the outer shaft of the implant catheter assembly 1611, and the handle 1616. The clasp actuation lines 624 can extend through and be axially movable relative to the handle 1616 and the outer shaft of the implant catheter assembly 1611. The clasp actuation lines 624 can also be axially movable relative to the actuation element 8102.

Referring to FIGS. 29 and 30, the width adjustment element 8211 can be releasably coupled to the coupler 8972 of the device 8200. Advancing and retracting the width adjustment element 8211 with the paddle width control 1628 widens and narrows the paddles. Advancing and retracting the actuation element 8102 with the paddle actuation control 1626 opens and closes the paddles of the device.

In the examples of FIGS. 29 and 30, the catheter or shaft of the implant catheter assembly 1611 is an elongate shaft extending axially between the proximal end portion 1622a, which is coupled to the handle 1616, and the distal end portion 1622b, which is coupled to the device 8200. The outer shaft of the implant catheter assembly 1611 can also include an intermediate portion 1622c disposed between the proximal and distal end portions 1622a, 1622b.

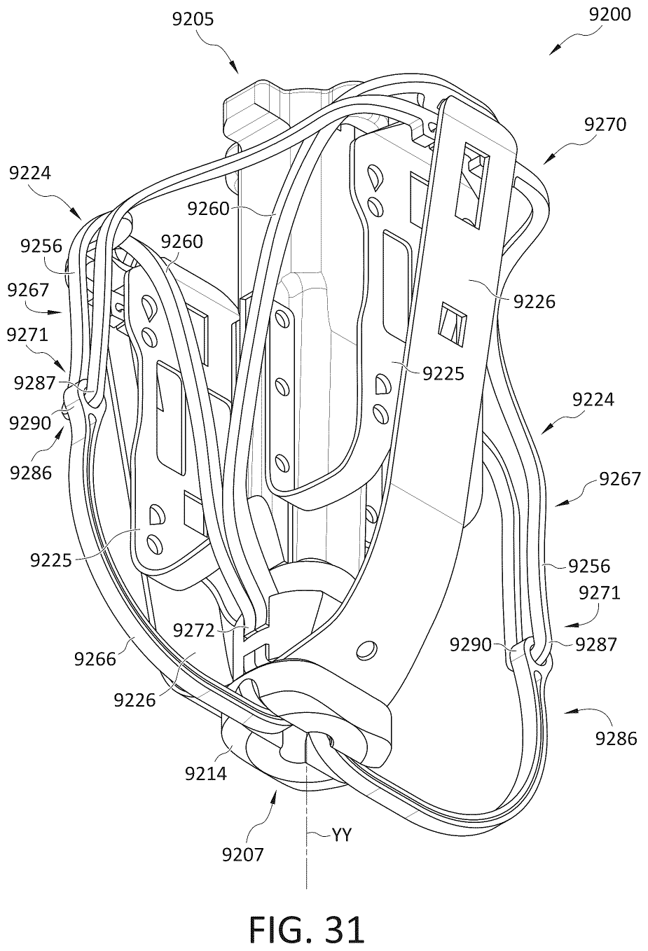

FIG. 31 illustrates an example valve repair system for repairing a native valve of a patient that the concepts of the present application can be applied to, which can include the same or similar features to other valve repair systems described. Referring to FIG. 31, the valve repair system can include an implantable valve repair device 9200 having a proximal or attachment portion 9205, paddle frames 9224, inner paddles 9225, outer paddles 9226, and a distal portion 9207. The proximal portion 9205, the distal portion 9207, and the paddle frames 9224 can be configured in a variety of ways.

The example valve repair device 9200 illustrated in FIG. 31 can be substantially similar to the valve repair device 8200 of FIG. 26. For example, the paddle frames 9224 can be symmetric along a longitudinal axis YY. However, in some implementations, the paddle frames 9224 are not symmetric about the longitudinal axis YY. The paddle frames 9224 include an outer frame member or members 9256 (e.g., wire frame portion, metal frame portion, etc.) and an optional inner frame member or members 9260 (e.g., wire frame portion, metal frame portion, etc.).

The outer frame members 9256 are flexibly attached to a connector 9266. Between the connector 9266 and the proximal portion 9205, the outer frame members 9256 form a curved shape. For example, in the illustrated example, the shape of the outer frame members 9256 resembles an apple shape in which the outer frame members 9256 are wider toward the proximal portion 9205 and narrower toward the distal portion 9207. In some implementations, however, the outer frame members 9256 can be otherwise shaped.

The inner frame members 9260 extend from the proximal portion 9205 toward the distal portion 9207. The inner frame members 9260 then extend inward to form retaining portions 9272 that are attached to an actuation cap 9214, in the same or similar manner as described in relation to any of the implantable valve repair device implementations described herein. The retaining portions 9272 and the actuation cap 9214 can be configured to attach in any suitable manner.

In some implementations, the inner frame members 9260 are rigid frame portions, while the outer frame members 9256 are flexible frame portions. The proximal end of the outer frame members 9256 connect to the proximal end of the inner frame members 9260 at, or adjacent, the proximal portion 9205 of the device.

The implantable valve repair device 9200 is configured to move the outer frame members 9256 from an expanded position to a narrowed position by pulling portions of the connector 9266 into the actuation cap 9214. The implantable valve repair device 9200 is also configured to move the inner frame members 9260 to open and close the paddles (e.g., outer paddles 320 and inner paddles 322 of FIG. 23) in accordance with some implementations disclosed herein.

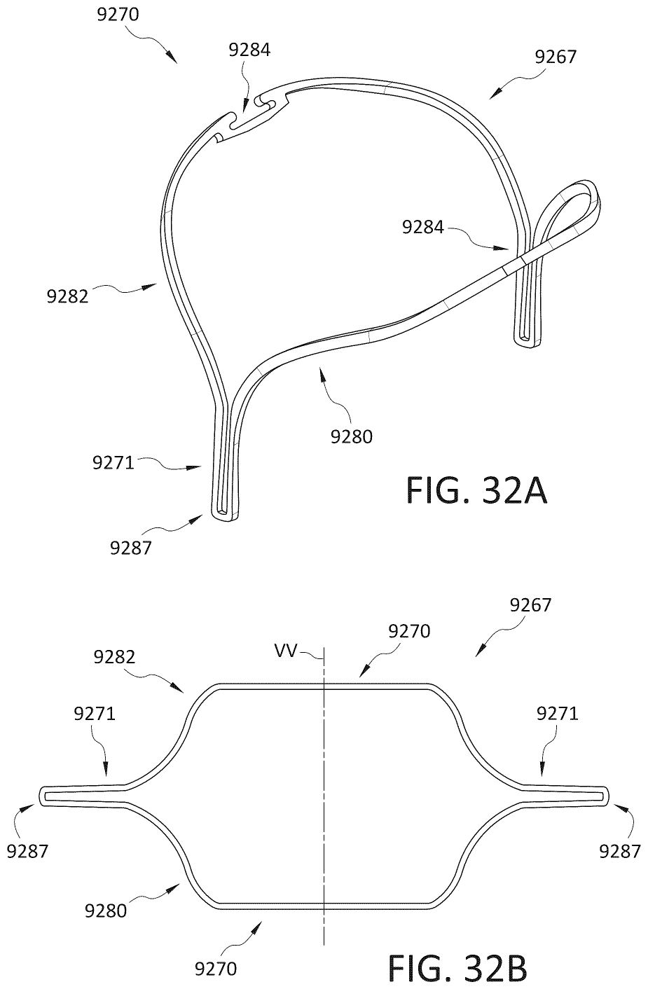

In the example valve repair device 8200 illustrated in FIG. 26, the outer frame members 8256 include a pair of adjustable width frame members 8967 (e.g., wire frame portion, metal frame portion, etc.), one arranged anteriorly and the other arranged posteriorly that are connected to the connector 8266 via suture joints. In some implementations, such as the example valve repair device 9200 of FIG. 31, the valve repair device 9200 includes an adjustable width frame member 9267 (e.g., wire frame, metal frame, etc.) that is formed as a single, integral piece, as illustrated in FIGS. 32-34, that connects to the connection portion 9266.

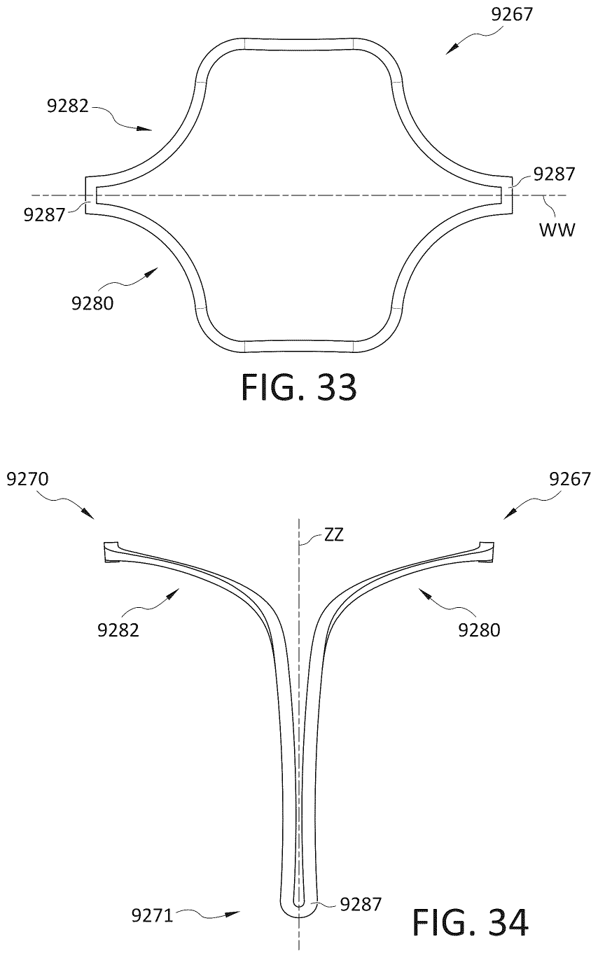

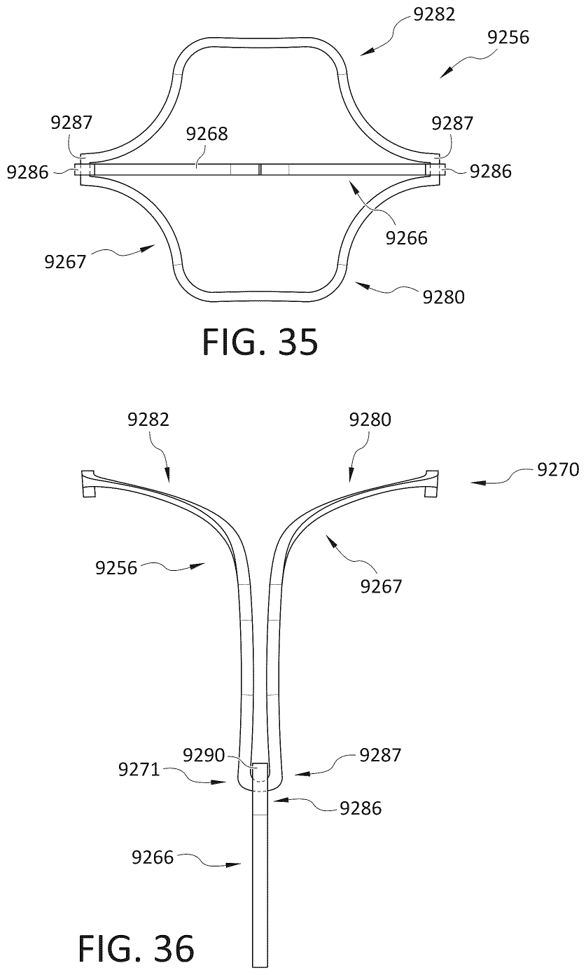

Adjustable width frame member 9267 (e.g., wire frame member, metal frame member, etc.) can be configured in a variety of ways, including different shapes and sizes and can be used with various valve repair systems/devices (e.g., the valve repair systems/devices of FIGS. 22-26 can be adapted to include adjustable width paddles and the frame member 9267). Referring to FIGS. 31-36, in the illustrated example, the adjustable width frame member 9267 includes a proximal portion 9270 and a distal portion 9271 opposite the proximal portion 9270. The adjustable width frame member 9267 includes an anterior portion 9280 and a posterior portion 9282 opposite the anterior portion 9280. In the illustrated example, the anterior portion 9280 is a mirror image of the posterior portion 9282 such that the adjustable width frame member 9267 is symmetric about axis WW (FIG. 33) and axis ZZ (FIG. 34). In other examples, however, the shape of the anterior portion 9280 can differ from the shape of the posterior portion 9282 such that the adjustable width frame member 9267 is not symmetric.

The proximal portion 9270 of each of the anterior portion 9280 and the posterior portion 9282, in addition to connecting to the proximal end of the inner frame member 9260 (e.g., wire frame portion, metal frame portion, etc.), can also include an opening 9284, as shown in FIG. 32, for receiving the paddles (e.g., the inner and outer paddles). As shown in FIGS. 32A and 34, the anterior portion 9280 and a posterior portion 9282 of the adjustable width frame member 9267 flare or taper away from each other toward the proximal portion 9270 such that distance between the anterior portion 9280 and the posterior portion 9282 is at a maximum at the proximal portion 9270.

The distal portion 9271 of the adjustable width frame member 9267 is connected to a proximal portion 9286 (FIG. 36) of the connector 9266. The distal portion 9271 of the adjustable width frame member 9267 can connect to a proximal portion 9286 of the connector 9266 in a variety of ways. In the illustrated example, the anterior portion 9280 and the posterior portion 9282 of the adjustable width frame member 9267 are integrally connected to each other at the distal portion 9271 to form a linking portion. As shown in FIGS. 32 and 34, the anterior portion 9280 and the posterior portion 9282 converge toward each other at the distal portion 9271 to form a pair of closed-ended projections 9287, one at the medial side and the other at the lateral side of the adjustable width frame member 9267.

The connector 9266 includes proximal portions 9286 adjacent a respective distal portion 9271 of the anterior portion 9280 and the posterior portion 9282 of the adjustable width frame member 9267. The proximal portion 9286 of connector 9266 includes an attachment portion 9290 for connecting to the distal portion 9271 of the adjustable width frame member 9267. The attachment portion 9290 can be configured in a variety of ways. In the illustrated example, each attachment portion 9290 includes a loop or hole through which a respective closed-ended projection 9287 passes through.

In the example of FIG. 31, the connector 9266 includes two or more layers of material. In some implementations, to connect one of the closed-ended projections 9287 to one of the attachment portions 9290, the layers can be separated to temporarily open the attachment portion 9290 at the proximal portion 9286 to accept the closed-ended projection 9287. For example, the closed hole shown in FIG. 31 can be temporarily opened. Once the closed-ended projections 9287 are properly positioned (e.g., the connector 9266 is centered relative to the adjustable width frame member 9267 or the closed-ended projections 9287 is coaxial with the connection portions 9266), the layers can be allowed to move back together to encircle the distal portion 9271. As a result, the attachment points between the adjustable width frame member 9267 and the connector 9266 act as hinges allowing for free rotation or pivoting between the adjustable width frame member 9267 and connector 9266 at the attachment point resulting in reduced stress and tension on the delivery system and device.

The adjustable width frame member 9267 can be formed as a single, integral piece in any suitable manner. For example, the adjustable width frame member 9267 can include a shape-memory alloy, such as Nitinol, to provide shape-setting capability. The adjustable width frame member 9267 can be shape-set into the as-assembled condition or assembled in a flat condition or profile. For example, referring to FIG. 32B, the adjustable width frame member 9267 is illustrated in a flat, planar condition. The adjustable width frame member 9267 can, for example, be laser cut into the flat, planar configuration and shape set, or otherwise bent or biased, about the axis VV (FIG. 32B) into the configuration illustrated in FIG. 32A. Thus, the closed-ended projections 9287 are created during formation of the adjustable width frame member 9267 and the adjustable width frame member 9267 bends at, or near, the proximal portion 9270 when deployed.

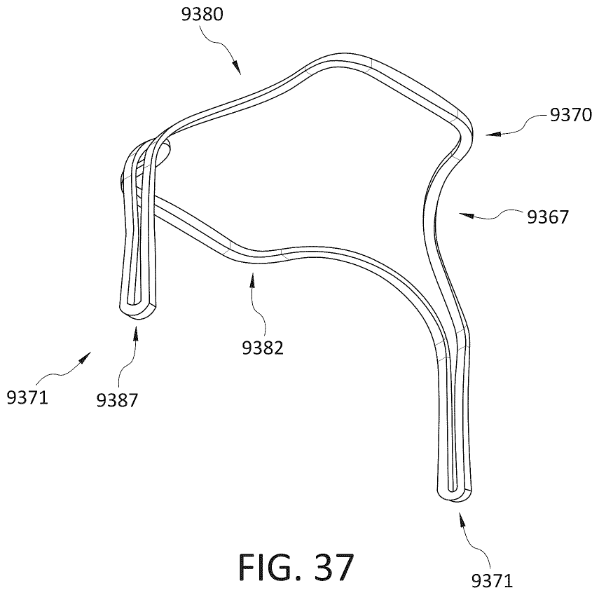

FIG. 37 illustrates an example of an adjustable width frame member 9367 useable as part of various valve repair systems or devices (e.g., valve repair device 9200, other valve repair systems and devices herein, etc.) for repairing a native valve of a patient, and the concepts of the present application can be applied thereto. The adjustable width frame member 9367 is formed as a single, integral piece that connects to a lower or distal frame member (e.g., connector 9266 of FIG. 31). The adjustable width frame member 9367 can be formed as a single, integral piece in any suitable manner. For example, the adjustable width frame member 9367 can include a shape-memory alloy, such as Nitinol, to provide shape-setting capability. The adjustable width frame member 9367 can be shape-set into the as-assembled condition or assembled in a flat condition or profile.

The adjustable width frame member 9367 (e.g., wire frame portion, metal frame portion, etc.) can be configured in a variety of ways, including different shapes and sizes. In the illustrated example of FIG. 37, the adjustable width frame member 9367 includes a proximal portion 9370 and a distal portion 9371 opposite the proximal portion 9370. The adjustable width frame member 9367 includes an anterior portion 9380 and a posterior portion 9382 opposite the anterior portion 9380. In the illustrated example, the anterior portion 9380 is a mirror image of the posterior portion 9382 such that the adjustable width frame member 9367 is symmetric. In other examples, however, the shape of the anterior portion 9380 can differ from the shape of the posterior portion 9382 such that the adjustable width frame member 9367 is not symmetric.

The anterior portion 9380 and a posterior portion 9382 of the adjustable width frame member 9367 extend away from each other toward the proximal portion 9370 such that distance between the anterior portion 9380 and the posterior portion 9382 is at a maximum at the proximal portion 9370. While the adjustable width frame member 9267 of FIG. 34 forms a Y-shape or V-shape with a flared proximal portion 9270 with a rounded profile, the shape of the adjustable width frame member 9367 of FIG. 37 forms a T-shape with a more squared profile than the proximal portion 9270.

The distal portion 9371 of the adjustable width frame member 9367 is configured to connect to a proximal end of a connector. The distal portion 9371 of the adjustable width frame member 9367 can connect to the connector in a variety of ways. In the illustrated example, the anterior portion 9380 and the posterior portion 9382 of the adjustable width frame member 9367 are integrally connected to each other at the distal portion 9371 to form a linking portion. As shown in FIG. 37, the anterior portion 9380 and the posterior portion 9382 converge toward each other at the distal portion 9371 to form a pair of closed-ended projections 9387, one at the medial side and the other at the lateral side of the adjustable width frame member 9367.

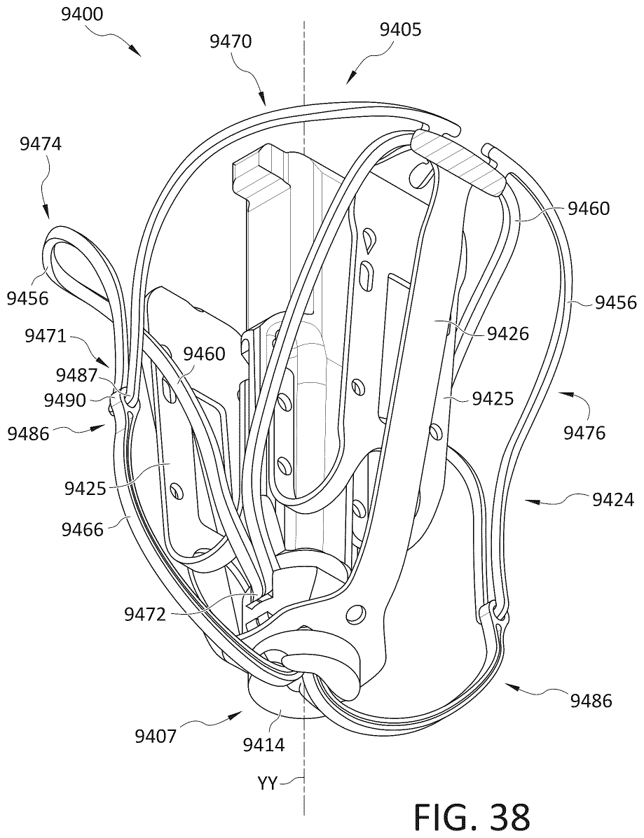

FIG. 38 illustrates an example of a valve repair system for repairing a native valve of a patient that the concepts of the present application can be applied to. Referring to FIG. 38, the valve repair system includes an implantable valve repair device 9400 having a proximal or attachment portion 9405, paddle frames 9424, inner paddles 9425, outer paddles 9426, and a distal portion 9407. The proximal portion 9405, the distal portion 9407, and the paddle frames 9424 can be configured in a variety of ways.

The example valve repair device 9400 illustrated in FIG. 31 can be similar to the valve repair device 9200 of FIG. 31. For example, the paddle frames 9424 can be symmetric along a longitudinal axis YY. However, in some implementations, the paddle frames 9424 are not symmetric about the longitudinal axis YY. The paddle frames 9424 include an outer frame member or members 9456 (e.g., wire frame portion, metal frame portion, etc.) and an inner frame member or members 9460 (e.g., wire frame portion, metal frame portion, etc.).

In some implementations, the outer frame members 9456 are flexibly attached to a connector 9466. In some implementations, between the connector 9466 and the proximal portion 9405, the outer frame members 9456 form a curved shape. For example, in the illustrated example, the shape of the outer frame members 9456 resembles an apple shape in which the outer frame members 9456 are wider toward the proximal portion 9405 and narrower toward the distal portion 9407. In some implementations, however, the outer frame members 9456 can be otherwise shaped.

The inner frame members 9460 extend from the proximal portion 9405 toward the distal portion 9407. The inner frame members 9460 then extend inward to form retaining portions 9472 that are attached to an actuation cap 9414, in the same or similar manner as described in relation to any of the implantable valve repair device implementations described herein. The retaining portions 9472 and the actuation cap 9414 can be configured to attach in any suitable manner.

In some implementations, the inner frame members 9460 are rigid frame portions, while the outer frame members 9456 are flexible frame portions. The proximal end of the outer frame members 9456 connect to the proximal end of the inner frame members 9460 at, or adjacent, the proximal portion 9405 of the device.

The implantable valve repair device 9400 is configured to move the outer frame members 9456 from an expanded position to a narrowed position by pulling portions of the connector 9466 into the actuation cap 9414. The implantable valve repair device 9400 is also configured to move the inner frame members 9460 to open and close the paddles (e.g., outer paddles 320 and inner paddles 322 of FIG. 23) in accordance with some implementations disclosed herein.

In the example valve repair device 8200 illustrated in FIG. 26, the outer frame members 8256 include a pair of adjustable width frame members 8967, one arranged anteriorly and the other arranged posteriorly, and a lower or distal frame member 8966 formed, at least in part, by the connection portions 8266, and connected to the connector(s) 8256 via suture joints. In the example valve repair device 9400 of FIGS. 38-42, however, the valve repair device 9400 includes a pair of adjustable width frame members 9467, one arranged medially and the other arranged laterally. In particular, the valve repair device 9400 includes a medial adjustable width frame member 9474 (e.g., wire frame portion, metal frame portion, etc.) and a lateral adjustable width frame member 9476 (e.g., wire frame portion, metal frame portion, etc.) which connect to a connector 9466.

The medial adjustable width frame member 9474 and the lateral adjustable width frame member 9476 can be formed in any suitable manner. For example, the medial adjustable width frame member 9474 and the lateral adjustable width frame member 9476 can include a shape-memory alloy, such as Nitinol, to provide shape-setting capability. Each of the medial adjustable width frame member 9474 and the lateral adjustable width frame member 9476 can be shape-set into the as-assembled condition or assembled in a flat condition or profile. In the illustrated example, the medial adjustable width frame member 9474 and the lateral adjustable width frame member 9476 are identical. Thus, the description below of the medial adjustable width frame member 9474 applies equally to the lateral adjustable width frame member 9476. In some implementations, however, the medial adjustable width frame member 9474 and the lateral adjustable width frame member 9476 can be configured differently.

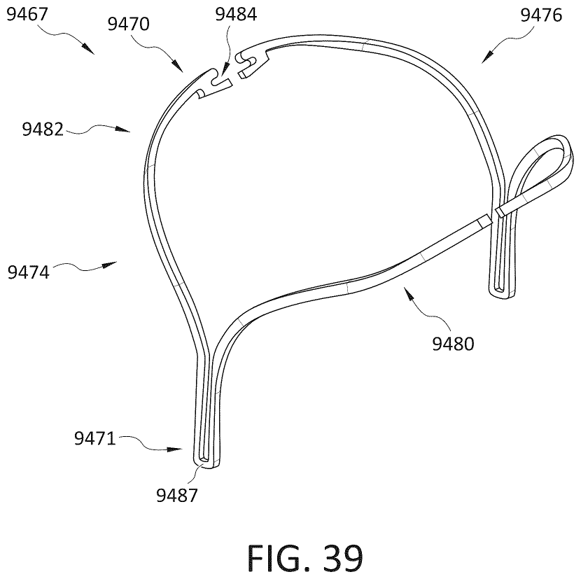

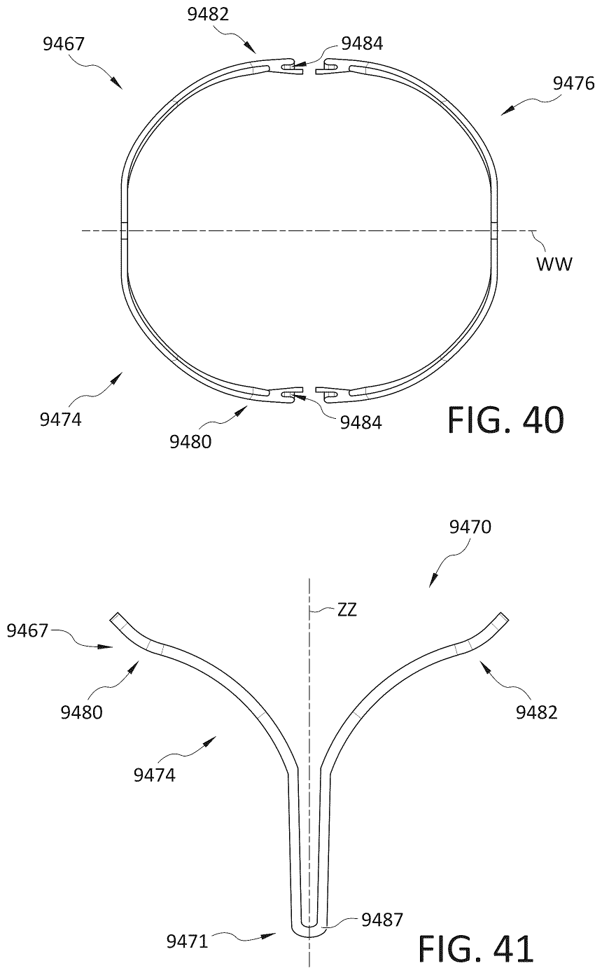

The medial adjustable width frame member 9474 can be configured in a variety of ways, including different shapes and sizes and can be used with various valve repair systems (e.g., the valve repair systems/devices of FIGS. 22-26 can be adapted to include adjustable width paddles and the frame member 9474). Referring to FIGS. 39-41, in the illustrated example, the medial adjustable width frame member 9474 includes a proximal portion 9470 and a distal end 9471 opposite the proximal portion 9470. The medial adjustable width frame member 9474 includes an anterior portion 9480 and a posterior portion 9482 opposite the anterior portion 9480. In the illustrated example, the anterior portion 9480 is a mirror image of the posterior portion 9482 such that the medial adjustable width frame member 9474 is symmetric about axis WW (FIG. 40) and axis ZZ (FIG. 41). In other examples, however, the shape of the anterior portion 9480 can differ from the shape of the posterior portion 9482 such that the medial adjustable width frame member 9474 is not symmetric.

The proximal portion 9470 of each of the anterior portion 9480 and the posterior portion 9482, in addition to connecting to the proximal end of the inner frame member 9460, can also include a recess or hole 9484, as shown in FIGS. 39-40, for receiving a portion of paddles (e.g., the inner and outer paddles). As best shown in FIG. 41, the anterior portion 9480 and a posterior portion 9482 of the adjustable width frame member 9467 flare or taper away from each other toward the proximal portion 9470 such that distance between the anterior portion 9480 and the posterior portion 9482 is at a maximum at the proximal portion 9470 and the medial adjustable width frame member 9474 forms a Y-shape or V-shape.

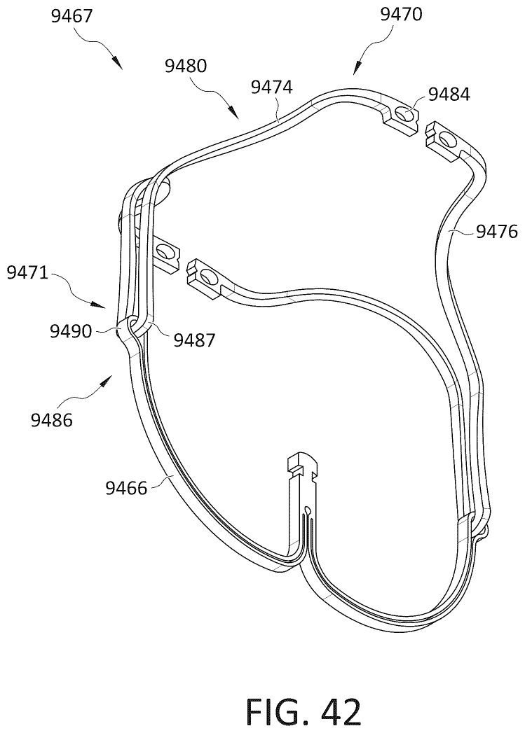

The distal end 9471 of the medial adjustable width frame member 9474 is connected to a proximal portion 9486 (FIGS. 38 and 42) of the connector 9466. The distal end 9471 of the medial adjustable width frame member 9474 can connect to the proximal portion 9486 of the connector 9468 in a variety of ways. In the illustrated example, the anterior portion 9480 and the posterior portion 9482 of the medial adjustable width frame member 9474 are integrally connected to each other at the distal end 9471 to form a linking portion. As shown in FIGS. 39 and 41, the anterior portion 9480 and the posterior portion 9482 converge toward each other at the distal end 9471 to form a closed-ended projection 9487.

The connector 9466 includes a proximal portion 9486 adjacent the distal end 9471 of the anterior portion 9480 and the posterior portion 9482 of the medial adjustable width frame member 9474. The proximal portion 9486 of connector 9466 includes an attachment portion 9490 for connecting to the distal end 9471 of the medial adjustable width frame member 9474. The attachment portion 9490 can be configured in a variety of ways. In the illustrated example, the attachment portion 9490 includes a loop or hole through which the closed-ended projection 9487 passes through. In another implementation, layers of the connector 9466 can be pulled apart to allow the frame members 9474, 9476 to be inserted into the attachment portion 9490. In this alternate implementation, the illustrated material bridge of the attachment portion 9490 is not included, so that the hole is not completely closed.

In the example of FIG. 38, the connector 9466 includes two or more layers of material. To connect the closed-ended projection 9487 to the attachment portions 9490, the layers can be separated to temporarily open the attachment portion 9490 at the proximal portion 9486 to accept the closed-ended projection 9487. Once the closed-ended projection 9487 is properly positioned (e.g., the connector 9466 is centered relative to the adjustable width frame member 9467 and/or the closed-ended projections 9487 are coaxial with the connector 9466), the layers can be allowed to move back together to encircle the distal end 9471.

The lateral adjustable width frame member 9476 can connect to the connector 9468 in the same manner as the medial adjustable width frame member 9474. As a result, the attachment points between the adjustable width frame member 9467 and the connector 9468 act as hinges allowing for free rotation or pivoting between the adjustable width frame member 9467 and connector 9468 at the attachment point resulting in reduced stress and tension on the delivery system and device.

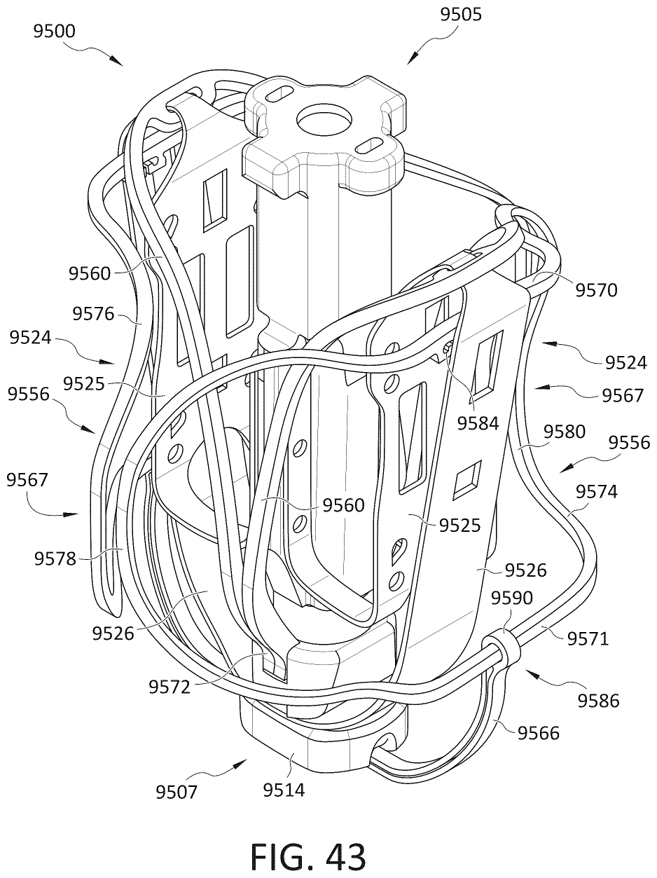

FIGS. 43-45 illustrate another example of one of the many valve repair systems for repairing a native valve of a patient that the concepts of the present application can be applied to. The valve repair system includes an implantable valve repair device 9500 having a proximal or attachment portion 9505, paddle frames 9524, inner paddles 9525, outer paddles 9526, and a distal portion 9507. The proximal portion 9505, the distal portion 9507, the paddle frames 9524, and the paddles 9525, 9526 can be configured in a variety of ways, such as any configuration disclosed herein.

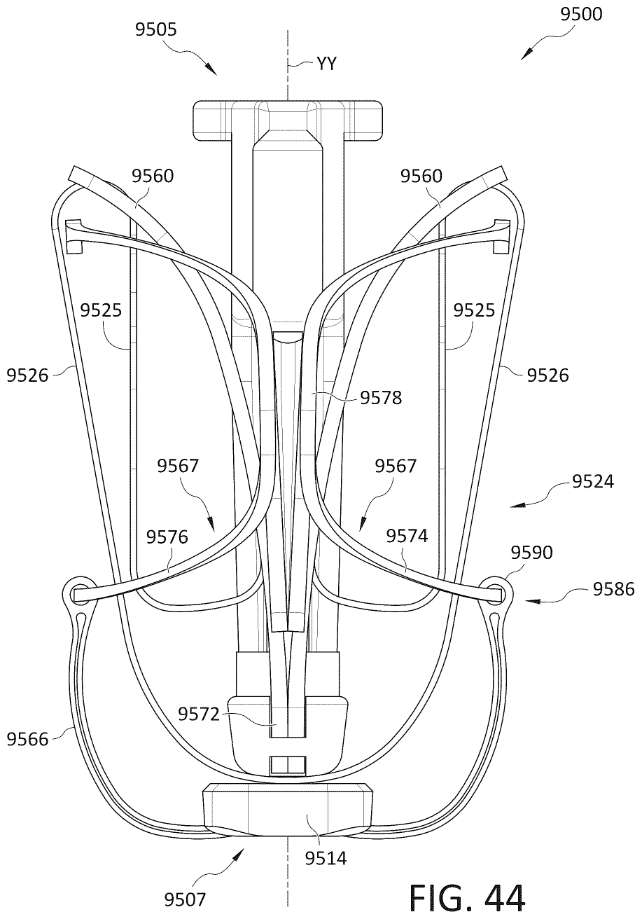

The example valve repair device 9500 illustrated in FIGS. 43-45 can be similar to the valve repair device 9200 of FIG. 31. For example, the paddle frames 9524, 9525 can be symmetric along a longitudinal axis YY (FIG. 44). However, in some implementations, the paddle frames 9524 are not symmetric about the longitudinal axis YY. The paddle frames 9524 include an outer frame member or members 9556 (e.g., wire frame portion, metal frame portion, etc.) and an inner frame member or members 9560 (e.g., wire frame portion, metal frame portion, etc.).

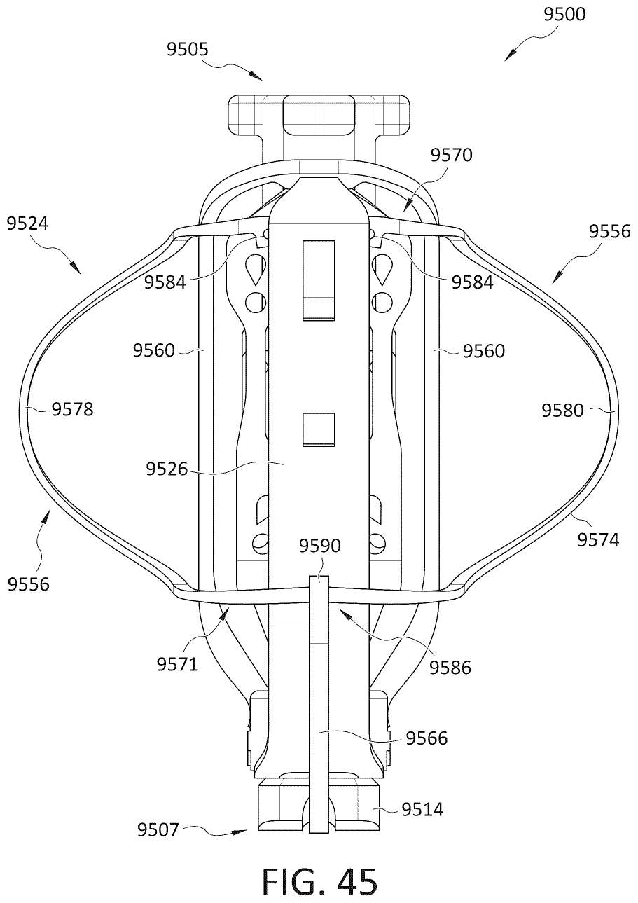

The outer frame members 9556 are flexibly attached to a connector 9566 at the distal portion 9507. Between the connector 9566 and the proximal portion 9505, the outer frame members 9556 form a curved shape. For example, in the illustrated example, the shape of the outer frame members 9556 resembles an oval shape as shown in FIG. 45. In some implementations, however, the outer frame members 9556 can be otherwise shaped.

The inner frame members 9560 extend from the proximal portion 9505 toward the distal portion 9507. The inner frame members 9560 then extend inward to form retaining portions 9572 that are attached to an actuation cap 9514, in the same or similar manner as described in relation to any of the implantable valve repair device implementations described herein. The retaining portions 9572 and the actuation cap 9514 can be configured to attach in any suitable manner.

In some implementations, the inner frame members 9560 are rigid frame portions, while the outer frame members 9556 are flexible frame portions. The proximal end of the outer frame members 9556 can connect to the proximal end of the inner frame members 9560 at, or adjacent, the proximal portion 9505 of the device. In some implementations, as shown in FIGS. 43-45, the proximal ends of the outer frame members 9556 and the proximal ends of the inner frame members 9560 are positioned to connect to the paddles 9525, 9526 at locations spaced apart from each other.

The implantable valve repair device 9500 is configured to move the outer frame members 9556 from an expanded position to a narrowed position by pulling portions of the connector 9566 into the actuation cap 9514. The implantable valve repair device 9500 is also configured to move the inner frame members 9560 to open and close the paddles 9525, 9526 in accordance with some implementations disclosed herein.

In the example valve repair device 9400 of FIGS. 38-42, the valve repair device 9400 includes the pair of adjustable width frame members 9467, one arranged medially and the other arranged laterally. Each of the pair of adjustable width frame members 9467 include an anterior portion 9480 and a posterior portion 9482 that are integrally connected to each other at the distal end 9471 to form the closed-ended projections 9487, which connect to connector 9466, one arranged medially and the other arranged laterally. In the example valve repair device 9500 of FIGS. 44-47, the valve repair device 9500 includes the pair of adjustable width frame members 9567, one arranged anteriorly and the other arranged posteriorly. In particular, the valve repair device 9500 includes an anterior adjustable width frame member 9574 and a posterior adjustable width frame member 9576, both of which connect to a connector 9566. In the valve repair device 9500, the connector 9566 is arranged to extend anteriorly and posteriorly as opposed to medially and laterally as with the valve repair device 9400 of FIGS. 38-42.

The anterior adjustable width frame member 9574 and the posterior adjustable width frame member 9576 can be formed in any suitable manner. For example, anterior adjustable width frame member 9574 and the posterior adjustable width frame member 9576 can include a shape-memory alloy, such as Nitinol, to provide shape-setting capability. Each of the anterior adjustable width frame member 9574 and the posterior adjustable width frame member 9576 can be shape-set into the as-assembled condition or assembled in a flat condition or profile. In the illustrated example, the anterior adjustable width frame member 9574 and the posterior adjustable width frame member 9576 are identical. Thus, the description below of the anterior adjustable width frame member 9574 applies equally to the posterior adjustable width frame member 9576. In some implementations, however, the anterior adjustable width frame member 9574 and the posterior adjustable width frame member 9576 can be configured differently.

The anterior adjustable width frame member 9574 (e.g., wire frame portion, metal frame portion, etc.) can be configured in a variety of ways, including different shapes and sizes. Referring to FIGS. 43-45, in the illustrated example, the anterior adjustable width frame member 9574 is formed as a closed loop having a proximal portion 9570, a distal portion 9571 opposite the proximal portion 9570, a medial portion 9578 extending between the proximal portion 9570 and the distal portion 9571, and a lateral portion 9580 extending between the proximal portion 9570 and the distal portion 9571 and opposite the medial portion 9578. In some implementations, however, the anterior adjustable width frame member 9574 can be open-ended, such as for example at the proximal portion 9570.

The proximal portion 9570 of the anterior adjustable width frame member 9574 can also include one or more recesses or holes 9584 (FIGS. 43 and 45) for receiving a portion of one of the paddles (e.g., the inner and outer paddles). The distal portion 9571 is connected to a proximal portion 9586 of the connector 9566 at, or adjacent, the midplane of the device 9500. The distal portion 9571 of the anterior adjustable width frame member 9574 can connect to the proximal portion 9586 of the connector 9568 in a variety of ways. In the illustrated example, the distal portion 9571 is a linear segment extending across the valve repair device 9500 between a medial side and a lateral side.

The connector 9566 includes a proximal portion 9586 adjacent the distal portion 9571 of the anterior adjustable width frame member 9574. The proximal portion 9586 includes an attachment portion 9590 for connecting to the distal portion 9571. The attachment portion 9590 can be configured in a variety of ways. In the illustrated example, the attachment portion 9590 includes a loop or hole through which the distal portion 9571 passes through.

The connector 9566 can include two or more layers of material. To connect the distal portion 9571 to the attachment portions 9590, the layers can be separated to temporarily open the attachment portion 9590 at the proximal portion 9586 to accept the distal portion 9571. Once the distal portion 9571 is properly positioned, the layers can be allowed to move back together to encircle the distal portion 9571. The illustrated material bridge and resulting closed hole is optional.

In the example FIGS. 43-45, when the distal portion 9571 of the anterior adjustable width frame member 9574 is attached to the connector 9566 by the attachment portion 9590 at the proximal portion 9586, the distal portion 9571 is positioned outward of the outer paddle 9526 (i.e., further anteriorly). In some implementations, however, the distal portion 9571 can be positioned inward of the outer paddle 9526 (i.e., located less anteriorly than the outer paddle 9526).

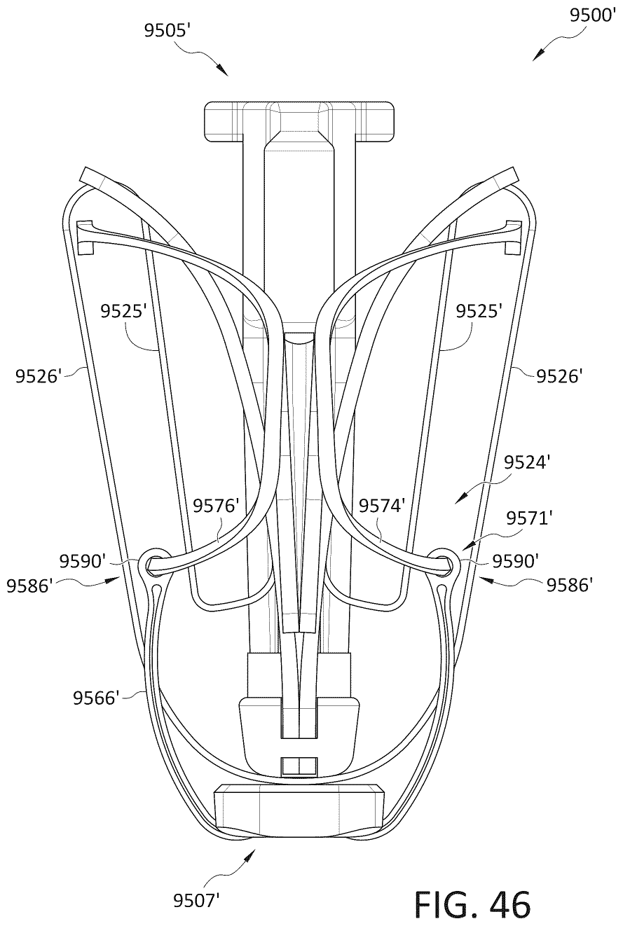

For example, FIG. 46 illustrates an example of an implantable valve repair device 9500′ having a proximal or attachment portion 9505′, paddle frames 9524′, inner paddles 9525′, outer paddles 9526′, and a distal portion 9507′. The valve repair device 9500′ can be configured substantially the same as the valve repair device 9500 of FIGS. 43-45. Thus, the description of the valve repair device 9500 applies equally to the valve repair device 9500′. For the valve repair device 9500′, however, when the distal portions 9571′ of the adjustable width frame members 9574′, 9576′ are attached to the proximal portions 9586′ of the connector 9566′ by the attachment portion 9590′, the distal portion 9571′ is positioned between the inner paddle 9525′ and the outer paddle 9526′ (i.e., to the anterior side of the inner paddle 9525′ and to the lateral side of the outer paddle 9526). The connector 9566′ extends through an opening or cutout in outer paddle 9526′. The connection portion 9590′ is also positioned between the inner paddle 9525′ and the outer paddle 9526′ (i.e., to the anterior side of the inner paddle 9525′ and to the lateral side of the outer paddle 9526). As shown in FIG. 46., the distance between the connecting portions 9566′ is less (i.e., a narrower spread) than the distance between the connecting portions 9566 of the device 9500 of FIGS. 43-45 (i.e., a wider spread).

Referring to FIGS. 43-45, the posterior adjustable width frame member 9576 can connect to the connector 9566 in the same manner as the anterior adjustable width frame member 9574. The attachment points between the adjustable width frame member 9567 and the connector 9566 act as hinges allowing for free rotation or pivoting between the adjustable width frame member 9567 and connector 9566 at the attachment point resulting in reduced stress and tension on the delivery system and device. Further, by arranging the attachment points between the adjustable width frame and the distal outer frame anteriorly and posteriorly, rather than medially and laterally, the attachment points do not restrict the depth to which the mitral valve leaflets can be inserted into the paddles. Thus, the leaflets can be fully received into the paddles.

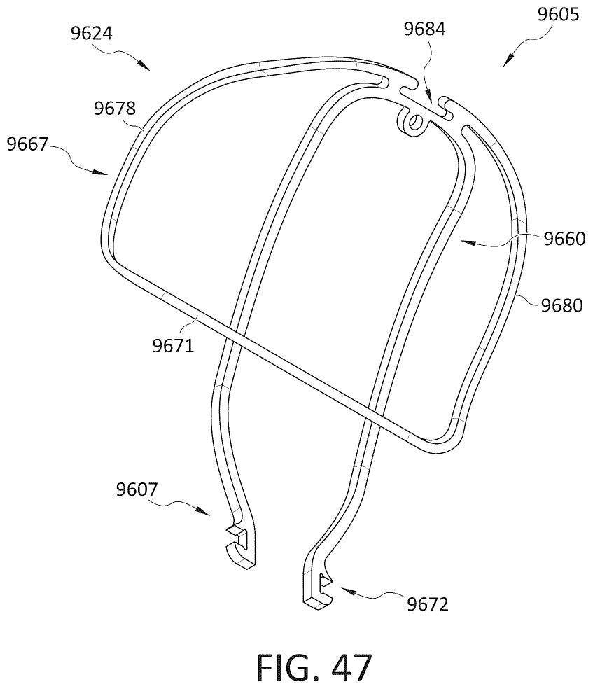

FIGS. 47-49 illustrate an example of a paddle frame 9624 that can be used with various valve repair systems and devices (e.g., valve repair device 9500, other valve repair systems and devices herein, etc.) for repairing a native valve of a patient that the concepts of the present application can be applied to. The paddle frame 9624 includes an adjustable width frame member or members 9667 and an inner frame member or members 9660 that are formed as a single, integral piece. The adjustable width frame member or members 9667 connects to a lower or distal frame member (e.g., connector 9566 of FIG. 43).

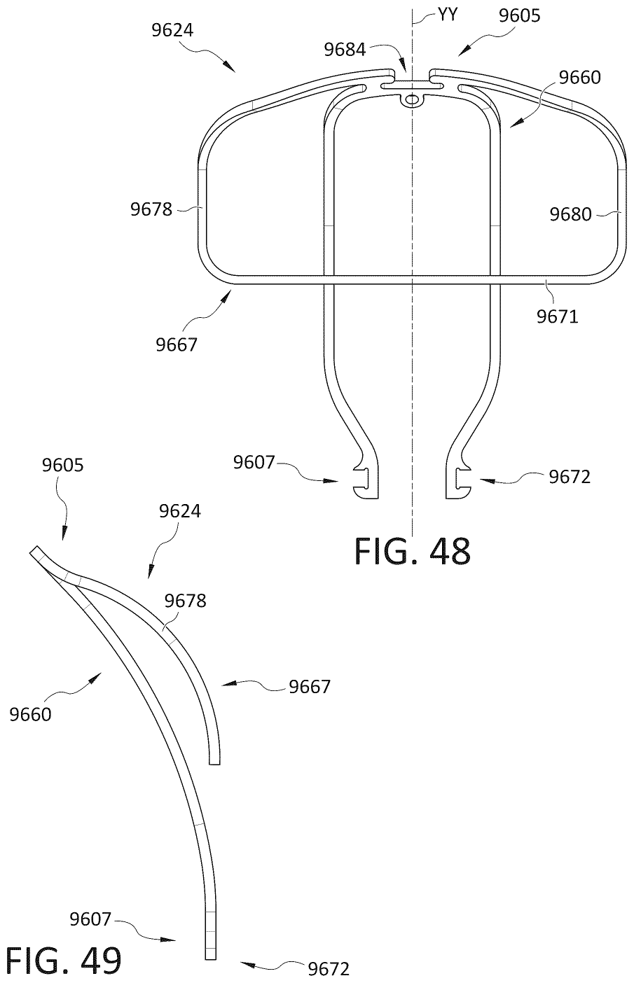

The paddle frame 9624 can be formed as a single, integral piece in any suitable manner. For example, the paddle frame 9624 can include a shape-memory alloy, such as Nitinol, to provide shape-setting capability. The paddle frame 9624 can be shape-set into the as-assembled condition or assembled in a flat condition or profile. The paddle frame 9624 can be symmetric along a longitudinal axis YY (FIG. 48). However, in some implementations, the paddle frames 9624 are not symmetric about the longitudinal axis YY. The paddle frame 9624 includes a proximal portion 9605 and a distal portion 9607 opposite the proximal portion 9605.

The inner frame members 9660 (e.g., wire frame portion, metal frame portion, etc.) extend from the proximal portion 9605 toward the distal portion 9607. The inner frame members 9660 at the distal portion 9607 extend inward to form retaining portions 9672 that are attached to an actuation cap (e.g., the actuation cap 9514 of FIG. 43), in the same or similar manner as described in relation to any of the implantable valve repair device implementations described herein. The retaining portions 9672 can be configured to attach in any suitable manner. In some implementations, the inner frame members 9660 are rigid frame portions, while the adjustable width frame members 9667 are flexible frame portions. The adjustable width frame members 9667 can integrally connect to the inner frame members 9660 at, or adjacent, the proximal portion 9605 of the paddle frame 9624. The proximal portion 9605 can also include one or more recesses or holes 9684 (FIGS. 47-48) for receiving a portion of one of the paddles (e.g., the inner and outer paddles).

The adjustable width frame members 9667 are flexibly attached to connection portions of a connector (e.g., the connection portions 9566 of the connector 9568 of FIGS. 43-45). The adjustable width frame member 9667 can be configured in a variety of ways, including different shapes and sizes. In the illustrated example, the adjustable width frame member 9667 is formed as a closed loop having a distal portion 9671 opposite the proximal portion 9605, a medial portion 9678 extending between the proximal portion 9505 and the distal portion 9671, and a lateral portion 9680 extending between the proximal portion 9605 and the distal portion 9671 and opposite the medial portion 9678.

The distal portion 9671 is configured to connect to a proximal end of a connector (e.g., the proximal portion 9586 of the connector 9566 of FIGS. 43-45) at, or adjacent, the midplane of the device. The distal portion 9671 can connect to the proximal end of the connector in a variety of ways. In the illustrated example, the distal portion 9671 is a linear segment extending across the valve repair device between a medial side and a lateral side and is configured to be received through a loop or hole in a connection portion in the same manner as the device of FIGS. 43-45.

Between the proximal portion 9605 and the distal portion 9671, the adjustable width frame members 9667 form a curved shape. For example, in the illustrated example, in the view illustrated in FIG. 478, the shape of the adjustable width frame members 9667 resembles a rectangle with the proximal portion 9605 curved or domed. In some implementations, however, the adjustable width frame members 9667 can be otherwise shaped.

The implantable valve repair device is configured to move the adjustable width frame members 9667 from an expanded position to a narrowed position by pulling portions of the connector into the actuation cap in the same manner as the connector 9566 and actuation cap 9514 of FIG. 43. The implantable valve repair device is also configured to move the inner frame members 9660 to open and close the paddles (e.g., outer paddles 320 and inner paddles 322 of FIG. 23) in accordance with some implementations disclosed herein.

The treatment techniques, methods, operations, steps, etc. described or suggested herein can be performed on a living subject (e.g., human, other animal, etc.) or on a non-living subject (e.g., a simulation, such as a cadaver, cadaver heart, simulator, anthropomorphic phantom, etc.). When performed on a simulation, the body parts, e.g., heart, tissue, valve, etc., can optionally be referred to as “simulated” (e.g., simulated heart, simulated tissue, simulated valve, etc.) and can comprise, for example, computerized and/or physical representations of body parts, tissue, etc.

Any of the various systems, assemblies, devices, apparatuses, etc. in this disclosure can be sterilized (e.g., with heat, radiation, ethylene oxide, hydrogen peroxide, etc.) to ensure they are safe for use with patients, and the methods herein can comprise (or additional methods comprise or consist of) sterilization of the associated system, device, apparatus, etc. (e.g., with heat, radiation, ethylene oxide, hydrogen peroxide, etc.).

While various inventive aspects, concepts and features of the disclosures can be described and illustrated herein as embodied in combination in the examples herein, these various aspects, concepts, and features can be used in many alternative examples, either individually or in various combinations and sub-combinations thereof. Unless expressly excluded herein all such combinations and sub-combinations are intended to be within the scope of the present application. Still further, while various alternative examples as to the various aspects, concepts, and features of the disclosures—such as alternative materials, structures, configurations, methods, devices, and components, alternatives as to form, fit, and function, and so on—may be described herein, such descriptions are not intended to be a complete or exhaustive list of available alternative examples, whether presently known or later developed. Those skilled in the art can readily adopt one or more of the inventive aspects, concepts, or features into additional examples and uses within the scope of the present application even if such examples are not expressly disclosed herein.

Additionally, even though some features, concepts, or aspects of the disclosures may be described herein as being a preferred arrangement or method, such description is not intended to suggest that such feature is required or necessary unless expressly so stated. Still further, example or representative values and ranges may be included to assist in understanding the present application, however, such values and ranges are not to be construed in a limiting sense and are intended to be critical values or ranges only if so expressly stated.

Moreover, while various aspects, features and concepts may be expressly identified herein as being inventive or forming part of a disclosure, such identification is not intended to be exclusive, but rather there may be inventive aspects, concepts, and features that are fully described herein without being expressly identified as such or as part of a specific disclosure, the disclosures instead being set forth in the appended claims. Descriptions of example methods or processes are not limited to inclusion of all steps as being required in all cases, nor is the order that the steps are presented to be construed as required or necessary unless expressly so stated. The words used in the claims have their full ordinary meanings and are not limited in any way by the description of the examples in the specification.

What is claimed is:

1. A valve repair device for repairing a native valve of a heart, the valve repair device comprising:
a pair of paddles movable between an open position and a closed position, wherein the pair of paddles are configured to attach the valve repair device to the native valve;
one or more paddle frames configured to open and close the pair of paddles, the one or more paddle frames comprising:
an inner frame portion extending from a proximal portion of the device to a distal portion of the device, the inner frame portion connected to the pair of paddles at the proximal portion;
an adjustable width frame portion having an anterior portion extending from the medial side of the device to the lateral side of the device and a posterior portion extending from the medial side of the device and the lateral side of the device opposite the anterior portion, wherein the anterior portion and the posterior portion are formed as a single frame member integrally joined at linking portions at the medial side of the device and a lateral side of the device;
a connector extending from the distal portion toward the proximal portion along both a medial side of the device and a lateral side of the device; and
wherein the connector includes attachment portions that connect to the linking portions to attach the adjustable width frame portion to the connector.

2. The valve repair device of claim 1, wherein the linking portions are configured as closed-ended projections.

3. The valve repair device of claim 2, wherein each of the attachment portions of the connector include a hole through which a respective one of the closed-ended projections is received.

4. The valve repair device of claim 1, wherein the anterior portion and the posterior portion of the adjustable width frame portion attach to one, or both, of the inner frame portion and the pair of paddles at the proximal portion of the device.

5. A valve repair device for repairing a native valve of a patient, the valve repair device comprising:
a pair of paddles movable between an open position and a closed position, wherein the pair of paddles are configured to attach the valve repair device to the native valve of the patient;
one or more paddle frames configured to open and close the pair of paddles, the one or more paddle frames comprising:
an inner frame portion extending from a proximal end of the device to a distal portion of the device, the inner frame portion connected to the pair of paddles at the proximal end;
a first adjustable width frame portion having a first anterior portion extending from the medial side of the device to adjacent a midline of the device and a first posterior portion extending from the medial side of the device to adjacent the midline of the device opposite the first anterior portion;
a second adjustable width frame portion having a second anterior portion extending from the lateral side of the device to adjacent the midline of the device and a second posterior portion extending from the lateral side of the device to adjacent the midline of the device opposite the second anterior portion;
wherein the first anterior portion and the first posterior portion are integrally joined at a first linking portion at the medial side of the device and the second anterior portion and the second posterior portion are integrally joined at a second linking portion at the lateral side of the device;
a connector extending from the distal portion toward the proximal end along both a medial side of the device and a lateral side of the device; and
wherein the connector includes a first attachment portion that connects to the first linking portion and a second attachment portion that connects to the second linking portion to attach the first and second adjustable width frame portions to the connector.

6. The valve repair device of claim 5, wherein the first and second linking portions are configured as closed-ended projections.

7. The valve repair device of claim 6, wherein the first and second attachment portions of the connector include a hole through which a respective one of the closed-ended projections is received.

8. The valve repair device of claim 5, wherein the first anterior portion and the first posterior portion attach to one, or both, of the inner frame portion and the pair of paddles at the proximal end of the device.

9. The valve repair device of claim 5, wherein the first adjustable width frame portion is symmetric.

10. A valve repair device for repairing a native valve of a patient, the valve repair device comprising:
a pair of paddles movable between an open position and a closed position, wherein the pair of paddles are configured to attach the valve repair device to the native valve of the patient;
one or more paddle frames configured to open and close the pair of paddles, the one or more paddle frames comprising:
an inner frame portion extending from a proximal portion of the device to a distal portion of the device, the inner frame portion connected to the pair of paddles at the proximal portion;
an anterior, adjustable width frame portion formed as a closed-loop extending from a medial side of the device to a lateral side of the device;
a posterior, adjustable width frame portion formed as a closed-loop extending from the medial side of the device to the lateral side of the device and opposite the anterior, adjustable width frame portion;
a connector extending from the distal portion toward the proximal portion along both an anterior side of the device and a posterior side of the device;
wherein the connector includes a first attachment portion that connects to an anterior distal portion of the anterior, adjustable width frame portion; and
wherein the connector includes a second attachment portion that connects to a posterior distal portion of the posterior, adjustable width frame portion.

11. The valve repair device of claim 10, wherein the anterior distal portion of the anterior, adjustable width frame portion and the posterior distal portion of the posterior, adjustable width frame portion comprise laterally-extending, linear sections.

12. The valve repair device of claim 11, wherein the first attachment portion of the connector includes a hole through which the posterior distal portion is received.

13. The valve repair device of claim 10, wherein the anterior, adjustable width frame portion and the posterior, adjustable width frame portion attach to one, or both, of the inner frame portion and the pair of paddles at the proximal portion of the device.

14. The valve repair device of claim 10, wherein each of the anterior, adjustable width frame portion and the posterior, adjustable width frame portion includes a shape-memory alloy.

15. The valve repair device of claim 10, wherein the anterior, adjustable width frame portion and the posterior, adjustable width frame portion are symmetric.

16. The valve repair device of claim 10, wherein the inner frame portion includes an anterior inner frame portion and a posterior inner frame portion separate from the anterior inner frame portion.

17. The valve repair device of claim 16, wherein the anterior inner frame portion is integrally formed with the anterior, adjustable width frame portion.

18. The valve repair device of claim 17, wherein the anterior inner frame portion is connected with the anterior, adjustable width frame portion adjacent the proximal portion of the device.

19. The valve repair device of claim 10, wherein a paddle of the pair of paddles comprises an inner paddle and an outer paddle, and wherein the first attachment portion connects to the anterior distal portion of the anterior, adjustable width frame portion at a position anteriorly outward of the outer paddle.

20. The valve repair device of claim 10, wherein a paddle of the pair of paddles comprises an inner paddle and an outer paddle, and wherein the first attachment portion connects to the posterior distal portion of the posterior, adjustable width frame portion at a position posteriorly outward of the inner paddle and posteriorly inward of the outer paddle.
